(19) United States
(12) Patent Application Publication
(10) Pub. No.: US 2009/0167161 A1
(43) Pub. Date: Jul. 2, 2009

(54) AROMATIC AMINE DERIVATIVES AND ORGANIC ELECTROLUMINESCENCE DEVICE USING THE SAME

(75) Inventors: Nobuhiro Yabunouchi, Chiba (JP); Masahiro Kawamura, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: IDEMITSU KOSAN CO., LTD., Chiyoda-ku (JP)

(21) Appl. No.: 12/198,497

(22) Filed: Aug. 26, 2008

(30) Foreign Application Priority Data
Dec. 28, 2007 (JP) 2007-341004
Dec. 28, 2007 (JP) 2007-341011

(51) Int. Cl. H01J 1/62; C07C 211/54; C07D 209/82

(52) U.S. Cl. 313/504; 564/308; 548/407

(57) ABSTRACT

Provided are an organic electroluminescence device and an aromatic amine derivative for realizing the device. The aromatic amine derivative improves the luminous efficiency of an organic electroluminescence device using the derivative, and its molecules hardly crystallize. The organic electroluminescence device has an organic thin film layer composed of one or a plurality of layers including at least a light emitting layer, the organic thin film layer being interposed between a cathode and an anode, and at least one layer of the organic thin film layer, especially a hole transporting layer contains the aromatic amine derivative alone or as a component of a mixture, so the organic electroluminescence device can be produced in improved yield, and has a long lifetime.

### TECHNICAL FIELD

The present invention relates to an aromatic amine derivative and an organic electroluminescence (EL) device using the same, and more particularly, to an aromatic amine derivative realizing the organic EL device capable of suppressing crystallization of a molecule in addition to allowing to improve a luminous efficiency, improving yields upon production of the organic EL device, and lengthening a lifetime of the organic EL device by using an asymmetric aromatic amine derivative having a specific structure as a hole transporting material.

### BACKGROUND ART

An organic EL device is a spontaneous light emitting device which utilizes such a principle that a fluorescent substance emits light by virtue of recombination energy of holes injected from an anode and electrons injected from a cathode by an application of an electric field. Since an organic EL device of the laminate type capable of being driven under low electric voltage has been reported by C. W. Tang et al. of Eastman Kodak Company (C. W. Tang and S. A. Vanslyke, Applied Physics Letters, Volume 51, Page 913, 1987, or the like), many studies have been conducted for an organic EL device using an organic material as a constituent material. Tang et al. used tris (8-quinolinolato) aluminum for a light emitting layer and a triphenyldiamine derivative for a hole transporting layer. Advantages of the laminate structure reside in the followings: an efficiency of the hole injection into the light emitting layer can be increased; an efficiency of forming exciton which are formed by blocking and recombining electrons injected from the cathode can be increased; and exciton formed within the light emitting layer can be enclosed. As described above, for the structure of the organic EL device, a two-layered structure having a hole transporting (injecting) layer and an electron transporting emitting layer and a three-layered structure having a hole transporting (injecting) layer, a light emitting layer, an electron transporting (injecting) layer, and the like are widely known. In order to increase the efficiency of recombination of injected holes and electrons in the devices of the laminate type, the device structure and the process for forming the device have been studied.

In general, when an organic EL device is driven or stored in an environment of high temperature, there occur adverse effects such as a change in the luminescent color, a decrease in emission efficiency, an increase in driving voltage, and a decrease in a lifetime of light emission. In order to prevent the adverse effects, it has been necessary that the glass transition temperature (Tg) of the hole transporting material be elevated. Therefore, it is necessary that many aromatic groups be held within a molecule of the hole transporting material (for example, an aromatic diamine derivative of Patent Document 1 and a fused aromatic ring diamine derivative of Patent Document 2), and in general, a structure having 8 to 12 benzene rings is preferably used.

However, when a large number of aromatic groups are present in a molecule, crystallization is liable to occur upon production of the organic EL device through the formation of a thin film by using those hole transporting materials. As a result, there arises a problem such as clogging of an outlet of a crucible to be used in vapor deposition or a reduction in yields of the organic EL device due to generation of defects of the thin film resulting from the crystallization. In addition, a compound having a large number of aromatic groups in any one of its molecules generally has a high glass transition temperature (Tg), but has a high sublimation temperature. Accordingly, there arises a problem in that the lifetime of the compound is short, because a phenomenon such as decomposition at the time of the vapor deposition or the formation of a nonuniform deposition film is expected to occur.

Meanwhile, there are some known documents each disclosing an asymmetric aromatic amine derivative. For example, Patent Document 3 describes an aromatic amine derivative having an asymmetric structure, but neither provides a specific example nor describes the characteristics of the asymmetric compound. In addition, Patent Document 4 describes, as an example, an asymmetric aromatic amine derivative having phenanthrene, but treats the asymmetric compound in the same way as that in the case of a symmetric compound, and does not describe the characteristics of the asymmetric compound at all. In addition, neither of those documents clearly describes a method of producing any such asymmetric compound in spite of the fact that a special synthesis method is needed for the asymmetric compound. Further, Patent Document 5 describes a method of producing an aromatic amine derivative having an asymmetric structure, but does not describe the characteristics of the asymmetric compound. Patent Document 6 describes an asymmetric compound having so high a glass transition temperature as to be thermally stable, but exemplifies merely a compound having carbazole.

In addition, for example, Patent Document 7 is a document about an amine compound having a spirobifluorene, but has no specific description concerning an asymmetric compound. In addition, the document has no description concerning a technology for combining carbazole and an amine compound.

As described above, the organic EL device having a high efficiency and a long lifetime has been reported, but it is yet hard to say that the device always shows sufficient performance, so development of the organic EL device having a further excellent performance has been strongly desired.

Patent Document 1: U.S. Pat. No. 4,720,432

Patent Document 2: U.S. Pat. No. 5,061,569

Patent Document 3: Japanese Patent Application Laid-Open No. 8-48656

Patent Document 4: Japanese Patent Application Laid-Open No. 11-135261

Patent Document 5: Japanese Patent Application Laid-Open No. 2003-171366

Patent Document 6: U.S. Pat. No. 6,242,115

Patent Document 7: Japanese Patent Application Laid-Open No. 7-278537

### DISCLOSURE OF THE INVENTION

### Problems to be Solved by the Invention

The present invention has been made with a view to solving the above-mentioned problems, and an object of the present invention is to provide and an organic EL device in which a molecule hardly crystallizes, and which can be produced with improved yields and has a long lifetime in addition to allowing to improve the luminous efficiency and lower the driving voltage, and an aromatic amine derivative realizing the organic EL device.

### Means for Solving the Problems

The inventors of the present invention have made extensive studies with a view toward achieving the above-mentioned object. As a result, the inventors have found that the above-mentioned problems can be solved by using a novel aromatic amine derivative having a specific substituent represented by the following general formula (1) as a material for an organic EL device, and particularly, as a hole transporting material, and thus the present invention has been completed.

Further, the inventors of the present invention have found that an amino group substituted by an aryl group represented by the general formulae (2) to (5) is suitable as an amine unit having a specific substituent. The inventors have found that because of being capable of interacting with electrodes, the amine unit is easy to inject charge, and has further effects of allowing low driving voltage owing to a high mobility, and as an interaction between molecules of the amine unit is small because of its steric hindrance, and the unit has such effects that crystallization is suppressed, yield in which an organic EL device is produced is improved, an organic EL device having a long lifetime can be provided, and particularly, a remarkably low driving voltage and long lifetime can be attained by combining a blue light emitting device. Further, when a compound having a large molecular weight has an asymmetric structure, the temperature at which the compound is deposited from the vapor can be lowered, so the decomposition of the compound at the time of the vapor deposition can be suppressed, and the lifetime of an organic EL device using the compound can be lengthened. That is, the present invention provides an aromatic amine derivative represented by the following general formula (1):

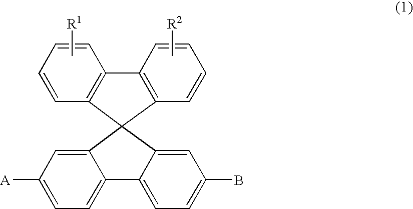

where R¹ and R² each independently represent a hydrogen atom, a substituted or unsubstituted aryl group having 6 to 50 ring atoms, or a substituted or unsubstituted alkyl group having 1 to 50 carbon atoms, and A and B are each independently represented by any one of the following general formulae (2) to (5) provided that A and B are different from each other:

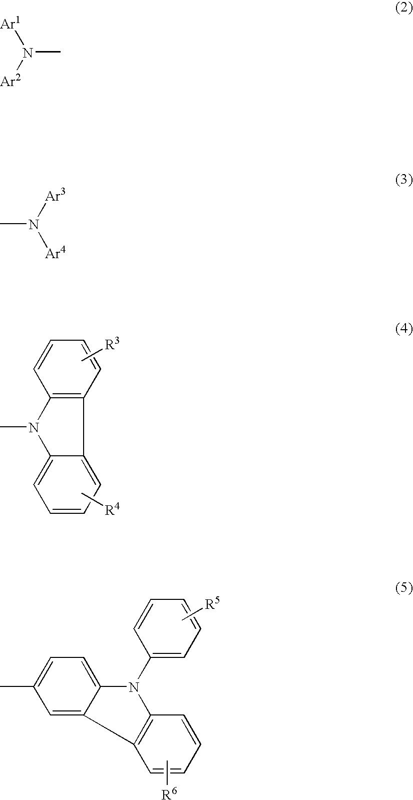

where Ar¹ to Ar⁴ each independently represent a substituted or unsubstituted aryl group having 6 to 50 ring atoms, and R³ to R⁶ each independently represent a hydrogen atom, a substituted or unsubstituted aryl group having 6 to 50 ring atoms, or a substituted or unsubstituted alkyl group having 1 to 50 carbon atoms.

Further, the present invention provides an organic EL device including an organic thin film layer formed of one or a plurality of layers including at least a light emitting layer and interposed between a cathode and an anode, in which at least one layer of the organic thin film layers contains the aromatic amine derivative alone or as a component of a mixture.

### Effect of the Invention

The aromatic amine derivative and the organic EL device using the same of the present invention hardly cause the crystallization of a molecule improves yields upon production of the organic device, and has a long lifetime in addition to allowing to lowering the driving voltage.

### BEST MODE FOR CARRYING OUT THE INVENTION

An aromatic amine derivative of the present invention is represented by the following general formula (1):

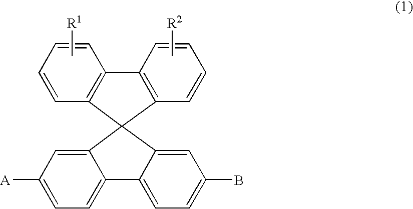

where R¹ and R² each independently represent a hydrogen atom, a substituted or unsubstituted aryl group having 6 to 50 ring atoms, or a substituted or unsubstituted alkyl group having 1 to 50 carbon atoms, and A and B are each independently represented by any one of the following general formulae (2) to (5) provided that A and B are different from each other:

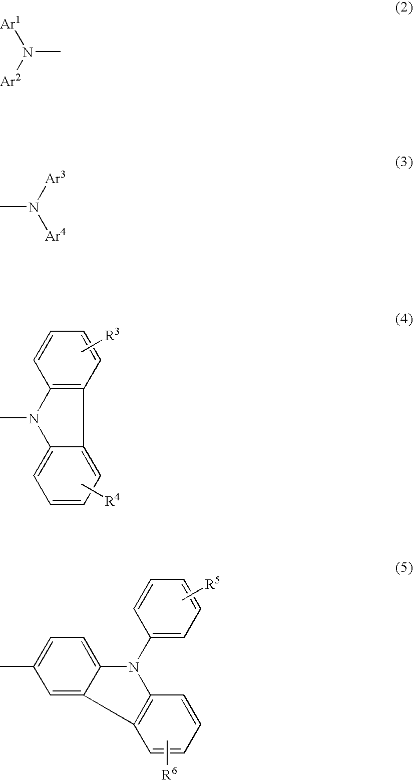

where Ar¹ to Ar⁴ in the general formulae (2) and (3) each independently represent a substituted or unsubstituted aryl group having 6 to 50 ring atoms, and R³ to R⁶ in the general formulae (4) and (5) each independently represent a hydrogen atom, a substituted or unsubstituted aryl group having 6 to 50 ring atoms, or a substituted or unsubstituted alkyl group having 1 to 50 carbon atoms.

Examples of the substituted or substituted aryl groups having 6 to 50 ring atoms represented by R¹ and R² in the general formula (1), represented by Ar¹ to Ar 4 in the general formulae (2) and (3), and represented by R³ to R⁶ in the general formulae (4) and (5) include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl4-yl group, a p-terphenyl3-yl group, a p-terphenyl2-yl group, an m-terphenyl4-yl group, an m-terphenyl3-yl group, an m-terphenyl-2-yl group, an o-tolyl group, an m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenylyl group, a 4″-t-butyl-p-terphenyl-4-yl group, a fluoranthenyl group, and a fluorenyl group.

Of those, preferred is a phenyl group, a naphthyl group, a biphenylyl group, a terphenylyl group, or a fluorenyl group.

The substituted or substituted alkyl groups having 1 to 50 carbon atoms represented by R¹ and R² in the general formula (1) and represented by R³ to R⁶ in the general formulae (4) and (5) may be straight or branched, and the alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a l-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, and a 2-norbornyl group.

The aromatic amine derivative of the present invention is preferably such that A in the general formula (1) represents a substituent represented by the general formula (2), B in the general formula (1) represents a substituent represented by the general formula (3), and at least three of the substituents represented by Ar¹ to Ar⁴ are different from one another.

The aromatic amine derivative of the present invention is preferably such that A in the general formula (1) represents a substituent represented by the general formula (2), B in the general formula (1) represents a substituent represented by the general formula (3), and three of the substituents represented by Ar¹ to Ar⁴ are identical to one another.

The aromatic amine derivative of the present invention is preferably such that A in the general formula (1) represents a substituent represented by the general formula (2), B in the general formula (1) represents a substituent represented by the general formula (3), Ar¹ and Ar² represent the same substituent, and Ar³ and Ar⁴ represent the same substituent.

The aromatic amine derivative of the present invention is preferably such that A in the general formula (1) represents a substituent represented by the general formula (2), B in the general formula (1) represents a substituent represented by the general formula (3), Ar¹ and Ar² each represent a biphenyl group, and Ar³ and Ar⁴ each independently represent a substituent selected from a phenyl group, a biphenyl group, a naphthyl group, a terphenyl group, and a fluorenyl group.

In addition, the aromatic amine derivative of the present invention is preferably such that A in the general formula (1) represents a substituent represented by the general formula (2) and B in the general formula (1) represents a substituent represented by the general formula (4). Further, in this case, it is preferable that Ar¹ and Ar² in the general formula (2) each independently represent a substituent selected from a phenyl group, a biphenyl group, a naphthyl group, a terphenyl group, and a fluorenyl group.

The aromatic amine derivative of the present invention is preferably such that A in the general formula (1) represents a substituent represented by the general formula (2) and B in the general formula (1) represents a substituent represented by the general formula (5) Further, in this case, it is preferable that Ar¹ and Ar2 in the general formula (2) each independently represent a substituent selected from a phenyl group, a biphenyl group, a naphthyl group, a terphenyl group, and a fluorenyl group.

The aromatic amine derivative of the present invention is preferably such that A in the general formula (1) represents a substituent represented by the general formula (4) and B in the general formula (1) represents a substituent represented by the general formula (5).

Specific examples of the aromatic amine derivative represented by the general formula (1) of the present invention are shown below. However, the derivative is not limited to these exemplified compounds.

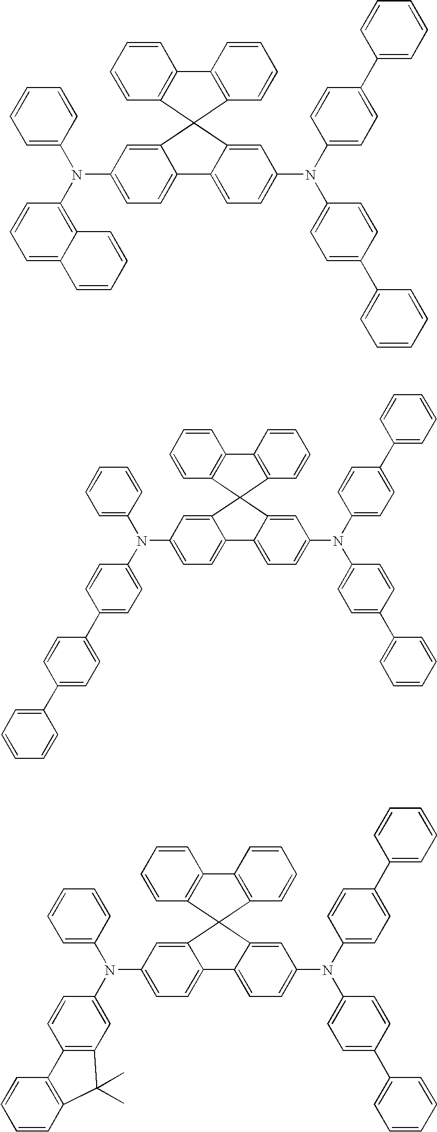

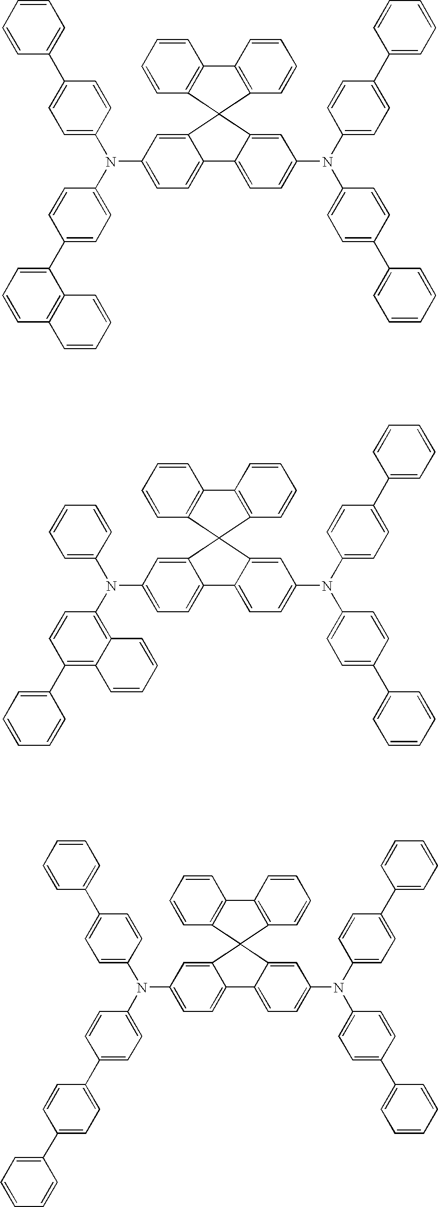

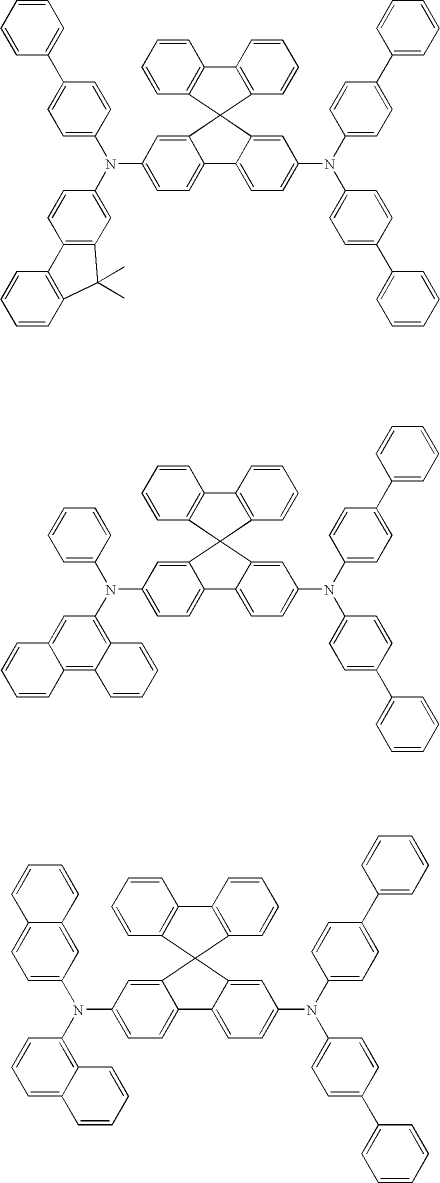

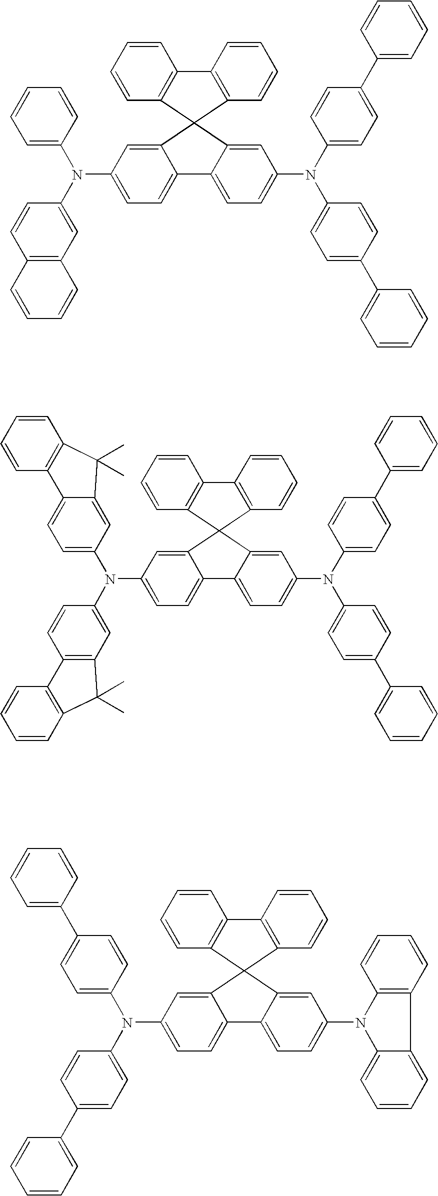

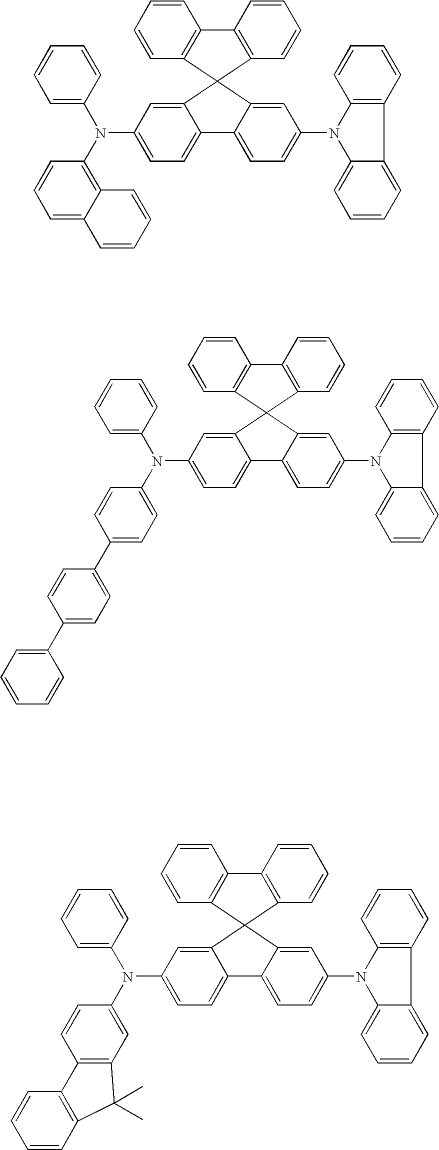

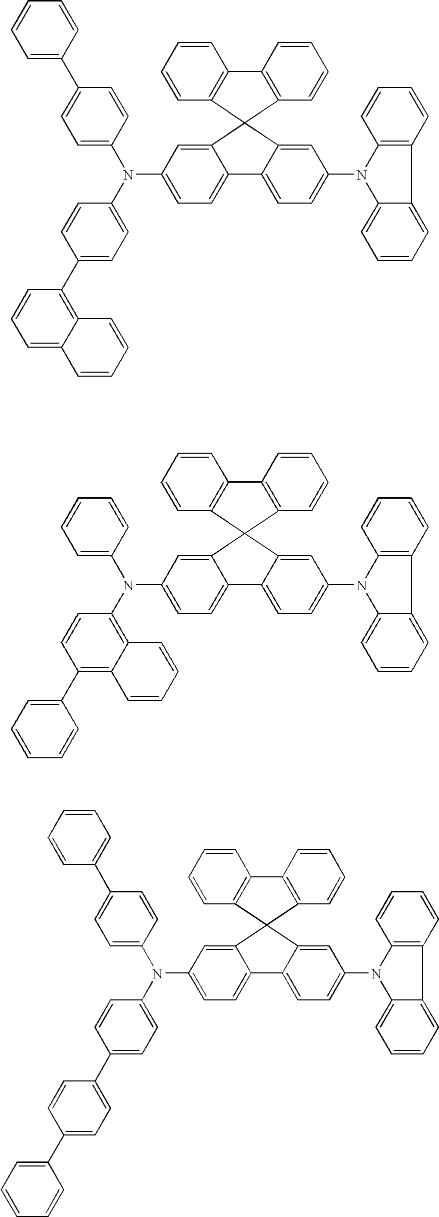

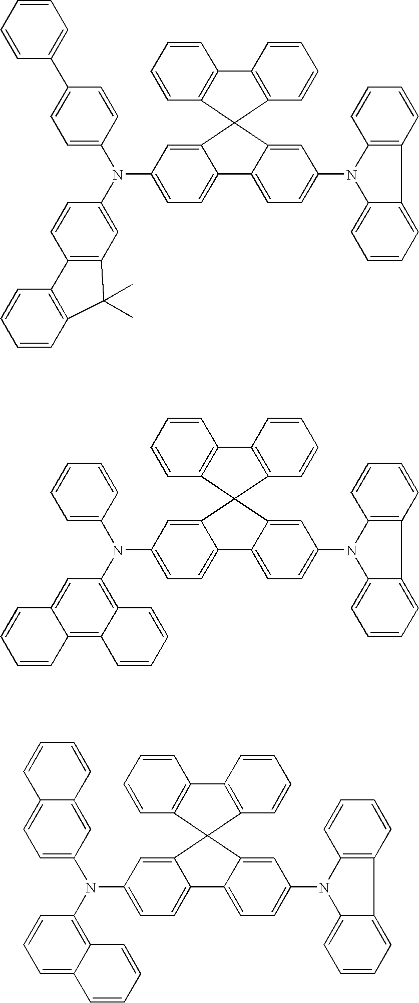

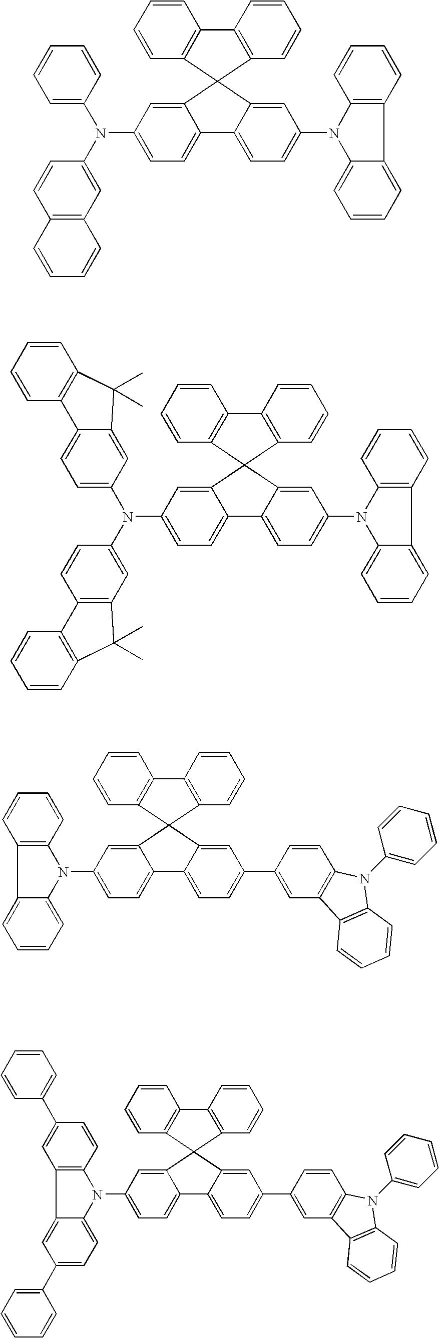

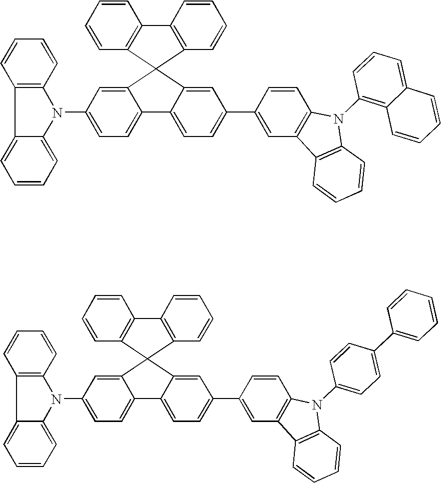

The aromatic amine derivative of the present invention is preferably a material for an organic electroluminescent device.

The aromatic amine derivative of the present invention is preferably a hole transporting material for an organic electroluminescent device.

The organic EL device of the present invention preferably includes an organic thin film layer formed of one or a plurality of layers including at least a light emitting layer and interposed between a cathode and an anode, in which at least one layer of the organic thin film layer contains the aromatic amine derivative alone or as a component of a mixture.

The organic EL device of the present invention is preferably such that the organic thin film layer has a hole transporting layer, and the hole transporting layer contains the aromatic amine derivative.

The organic EL device of the present invention is preferably such that the organic thin film layer has a plurality of hole transporting layers, and a layer in direct contact with the light emitting layer contains the aromatic amine derivative.

In addition, the organic EL device of the present invention is preferably such that the light emitting layer contains a styrylamine compound and/or an arylamine compound.

Examples of the styrylamine compound include compounds each represented by the following general formula (I), and examples of the arylamine compound include compounds each represented by the following general formula (II):

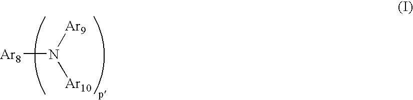

where: Ar₈ represents a group selected from phenyl, biphenyl, terphenyl, stilbene, and distyrylaryl groups; Ar₉ and Ar₁₀ each represent a hydrogen atom or an aromatic group having 6 to 20 carbon atoms, and each of Ar₉ and Ar₁₀ may be substituted; p′ represents an integer of 1 to 4; and Ar₉ and/or Ar₁₀ are/is more preferably substituted by styryl groups/a styryl group.

Here, the aromatic group having 6 to 20 carbon atoms is preferably a phenyl group, a naphthyl group, an anthranyl group, a phenanthryl group, a terphenyl group, or the like.

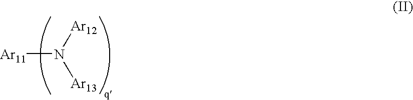

where: Ar₁₁ to Ar₁₃ each represent an aryl group having 5 to 40 ring carbon atoms and which may be substituted; and q′ represents an integer of 1 to 4.

Here, examples of the aryl group having 5 to 40 ring atoms preferably include phenyl, naphthyl, anthranyl, phenanthryl, pyrenyl, coronyl, biphenylyl, terphenylyl, pyrrolyl, furanyl, thiophenyl, benzothiophenyl, oxadiazolyl, diphenylanthranyl, indolyl, carbazolyl, pyridyl, benzoquinolyl, fluoranthenyl, acenaphthof luoranthenyl, and stilbene. In addition, the aryl group having 5 to 40 ring atoms may be further substituted by a substituent. Examples of the substituent preferably include: an alkyl group having 1 to 6 carbon atoms such as an ethyl group, a methyl group, an isopropyl group, an n-propyl group, an s-butyl group, a t-butyl group, a pentyl group, a hexyl group, a cyclopentyl group, or a cyclohexyl group; an alkoxy group having 1 to 6 carbon atoms such as an ethoxy group, a methoxy group, an isopropoxy group, an n-propoxy group, an s -butoxy group, a t-butoxy group, a pentoxy group, a hexyloxy group, a cyclopentoxy group, or a cyclohexyloxy group; an aryl group having 5 to 40 ring atoms; an amino group substituted by an aryl group having 5 to 40 ring atoms; an ester group containing an aryl group having 5 to 40 ring atoms; an ester group containing an alkyl group having 1 to 6 carbon atoms; a cyano group; a nitro group; and a halogen atom such as chlorine, bromine, and iodine.

The organic EL device of the present invention is preferably such that the organic thin film layer has a plurality of hole injecting and transporting layers, and at least one of the layers is a layer containing an acceptor material.

The aromatic amine derivative of the present invention is particularly preferably used in an organic EL device that emits blue-based light.

The structure of the organic EL device of the present invention is described in the following.

(1) Organic EL Device Constitution

Typical examples of the constitution of the organic EL device of the present invention include the following:

(1) an anode/light emitting layer/cathode;

(2) an anode/hole injecting layer/light emitting layer/cathode;

(3) an anode/light emitting layer/electron injecting layer/cathode;

(4) an anode/hole injecting layer/light emitting layer/electron injecting layer/cathode;

(5) an anode/organic semiconductor layer/light emitting layer/cathode;

(6) an anode/organic semiconductor layer/electron blocking layer/light emitting layer/cathode;

(7) an anode/organic semiconductor layer/light emitting layer/adhesion improving layer/cathode;

(8) an anode/hole injecting layer/hole transporting layer/light emitting layer/electron injecting layer/cathode;

(9) an anode/insulating layer/light emitting layer/insulating layer/cathode;

(10) an anode/inorganic semiconductor layer/insulating layer/light emitting layer/insulating layer/cathode;

(11) an anode/organic semiconductor layer/insulating layer/light emitting layer/insulating layer/cathode;

(12) an anode/insulating layer/hole injecting layer/hole transporting layer/light emitting layer/insulating layer/cathode; and

(13) an anode/insulating layer/hole injecting layer/hole transporting layer/light emitting layer/electron injecting layer/cathode.

Of those, the constitution (8) is preferably used in ordinary cases. However, the constitution is not limited to the foregoing.

The aromatic amine derivative of the present invention may be used in any one of the organic thin film layers of the organic EL device. The derivative can be used in a light emitting zone or a hole transporting zone. The derivative is used preferably in the hole transporting zone, or particularly preferably in a hole injecting layer, thereby making a molecule hardly crystallize and improving yields upon production of the organic EL device.

The amount of the aromatic amine derivative of the present invention to be incorporated into the organic thin film layers is preferably 30 to 100 mol %.

(2) Light-Transmissive Substrate

The organic EL device of the present invention is prepared on a light-transmissive substrate. Here, the light-transmissive substrate is the substrate which supports the organic EL device. It is preferable that the light-transmissive substrate have a transmittance of light of 50% or higher in the visible region of 400 to 700 nm and be flat and smooth.

Examples of the light-transmissive substrate include glass plates and polymer plates. Specific examples of the glass plate include plates formed of soda-lime glass, glass containing barium and strontium, lead glass, aluminosilicate glass, borosilicate glass, barium borosilicate glass, and quartz. Specific examples of the polymer plate include plates formed of polycarbonate, acrylic, polyethylene terephthalate, polyether sulfide, and polysulfone.

(3) Anode

The anode of the organic EL device of the present invention has the function of injecting holes into the hole transporting layer or the light emitting layer. It is effective that the anode has a work function of 4.5 eV or higher. Specific examples of the material for the anode used in the present invention include indium tin oxide (ITO) alloys, tinoxide (NESA), indiumzincoxide (IZO), gold, silver, platinum, and copper.

The anode can be prepared by forming a thin film of the electrode material described above in accordance with a process such as the vapor deposition process and the sputtering process.

When the light emitted from the light emitting layer is obtained through the anode, it is preferable that the anode have a transmittance of the emitted light higher than 10%. It is also preferable that the sheet resistivity of the anode be several hundred Ω/□ or smaller. The thickness of the anode is, in general, selected in the range of 10 nm to 1 μm and preferably in the range of 10 to 200 nm although the preferable range may be different depending on the used material.

(4) Light Emitting Layer

The light emitting layer of the organic EL device has a combination of the following functions (1) to (3).

(1) The injecting function: the function of injecting holes from the anode or the hole injecting layer and injecting electrons from the cathode or the electron injecting layer when an electric field is applied.

(2) The transporting function: the function of transporting injected charges (i.e., electrons and holes) by the force of the electric field.

(3) The light emitting function: the function of providing the field for recombination of electrons and holes and leading to the emission of light.

However, the easiness of injection may be different between holes and electrons and the ability of transportation expressed by the mobility may be different between holes and electrons. It is preferable that one of the charges be transferred.

A known method such as a vapor deposition method, a spin coating method, or an LB method is applicable to the formation of the light emitting layer. The light emitting layer is particularly preferably a molecular deposit film. The term “molecular deposit film” as used herein refers to a thin film formed by the deposition of a material compound in a vapor phase state, or a film formed by the solidification of a material compound in a solution state or a liquid phase state. The molecular deposit film can be typically distinguished from a thin film formed by the LB method (molecular accumulation film) on the basis of differences between the films in aggregation structure and higher order structure, and functional differences between the films caused by the foregoing differences.

In addition, as disclosed in Japanese Patent Application Laid-Open No. 57-51781, the light emitting layer can also be formed by: dissolving a binder such as a resin and a material compound in a solvent to prepare a solution; and forming a thin film from the prepared solution by the spin coating method or the like.

In the present invention, where desired, the light emitting layer may include other known light emitting materials other than the light emitting material composed of the aromatic amine derivative of the present invention, or a light emitting layer including other known light emitting material may be laminated to the light emitting layer including the light emitting material composed of the aromatic amine derivative of the present invention as long as the object of the present invention is not adversely affected.

Examples of the light emitting material or the doping material which can be used in the light emitting layer together with the aromatic amine derivative of the present invention include, but not limited to, anthracene, naphthalene, phenanthrene, pyrene, tetracene, coronene, chrysene, fluoresceine, perylene, phthaloperylene, naphthaloperylene, perynone, phthaloperynone, naphthaloperynone, diphenylbutadiene, tetraphenylbutadiene, coumarin, oxadiazole, aldazine, bisbenzoxazoline, bisstyryl, pyrazine, cyclopentadiene, quinoline metal complexes, aminoquinoline metal complexes, benzoquinoline metal complexes, imine, diphenylethylene, vinylanthracene, diaminocarbazole, pyrane, thiopyrane, polymethine, merocyanine, imidazole-chelated oxynoid compounds, quinacridone, rubrene, and fluorescent dyes.

A host material that can be used in a light emitting layer together with the aromatic amine derivative of the present invention is preferably a compound represented by any one of the following formulae (i) to (ix):

an asymmetric anthracene represented by the following general formula (i):

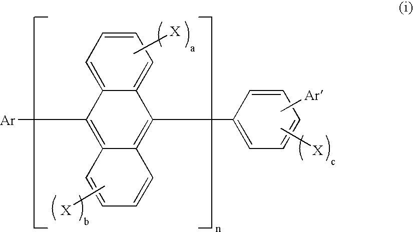

where: Ar represents a substituted or unsubstituted fused aromatic group having 10 to 50 ring carbon atoms;

Ar′ represents a substituted or unsubstituted aromatic group having 6 to 50 ring carbon atoms;

X represents a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a carboxyl group, a halogen atom, a cyano group, a nitro group, or a hydroxyl group;

a, b, and c each represent an integer of 0 to 4; and

n represents an integer of 1 to 3, and when n represents 2 or more, anthracene nuclei in [] may be identical to or different from each other;

an asymmetric monoanthracene derivative represented by the following general formula (ii):

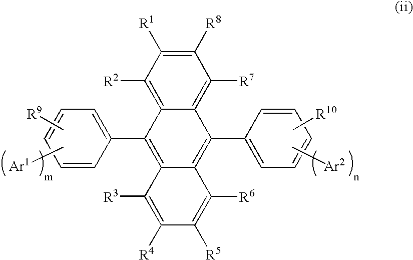

where: Ar¹ and Ar² each independently represent a substituted or unsubstituted aromatic ring group having 6 to 50 ring carbon atoms; m and n each represent an integer of 1 to 4; provided that Ar¹ and Ar² are not identical to each other when m =n=1 and positions at which Ar¹ and Ar² are bound to a benzene ring are bilaterally symmetric, and m and n represent different integers when m or n represents an integer of 2 to 4; and

R¹ to R¹⁰ each independently represent a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group;

an asymmetric pyrene derivative represented by the following general formula (iii):

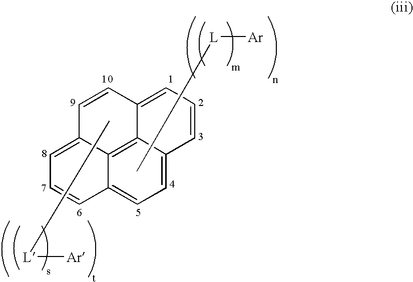

where: Ar and Ar′ each represent a substituted or unsubstituted aromatic group having 6 to 50 ring carbon atoms;

L and L′ each represent a substituted or unsubstituted phenylene group, a substituted or unsubstituted naphthalenylene group, a substituted or unsubstituted fluorenylene group, or a substituted or unsubstituted dibenzosilolylene group;

m represents an integer of 0 to 2; n represents an integer of 1 to 4; s represents an integer of 0 to 2; t represents an integer of 0 to 4; and

in addition, L or Ar binds to any one of 1- to 5-positions of pyrene, and L′ or Ar′ binds to any one of 6- to 10-positions of pyrene,

provided that Ar, Ar′, L, and L′ satisfy the following item (1) or (2) when n+t represents an even number:
(1) Ar≠Ar′ and/or L≠L′ (where the symbol “≠” means that groups connected with the symbol have different structures); and
(2) when Ar=Ar′ and L=L′,
   (2-1) m≠s and/or n≠t, or
   (2-2) when m=s and n=t,
      (2-2-1) in the case where L and L′ (or pyrene) bind (or binds) to different binding positions on Ar and Ar′, or (2-2-2) in the case where L and L′ (or pyrene) bind (or binds) to the same binding positions on Ar and Ar′, the case where the substitution positions of L and L′, or of Ar and Ar′ in pyrene are 1- and 6-positions, or 2- and 7-positions does not occur;

an asymmetric anthracene derivative represented by the following general formula (iv):

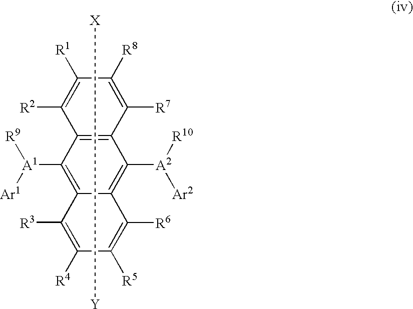

where: A¹ and A² each independently represent a substituted or unsubstituted fused aromatic ring group having 10 to 20 ring carbon atoms;

Ar¹ and Ar² each independently represent a hydrogen atom, or a substituted or unsubstituted aromatic ring group having 6 to 50 ring carbon atoms;

R¹ to R¹⁰ each independently represent a hydrogen atom, a substituted or unsubstituted aromatic ring group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group; and

the number of each of Ar¹, Ar², R⁹, and R¹⁰ may be two or more, and adjacent groups may form a saturated or unsaturated cyclic structure,

provided that the case where groups symmetric with respect to the X-Y axis shown on central anthracene in the general formula (1) bind to 9- and 10-positions of the anthracene does not occur;

an anthracene derivative represented by the following general formula (v):

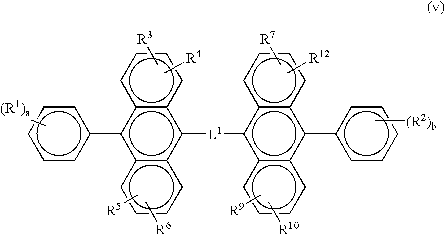

where: R¹ to R¹⁰ each independently represent a hydrogen atom, an alkyl group, a cycloalkyl group, an aryl group which may be substituted, an alkoxyl group, an aryloxy group, an alkylamino group, an alkenyl group, an arylamino group, or a heterocyclic group which may be substituted; a and b each represent an integer of 1 to 5, and, when a or b represents 2 or more, R¹'s or R²'s may be identical to or different from each other, or R¹'s or R²'s may be bonded to each other to form a ring; R³ and R⁴, R⁵ and R⁶, R⁷ and R⁸ , or R⁹ and R¹⁰ may be bonded to each other to form a ring; and L¹ represents a single bond, —O—, —S—, —N(R)— (where R represents an alkyl group or an aryl group which may be substituted), an alkylene group, or an arylene group;

an anthracene derivative represented by the following general formula (vi):

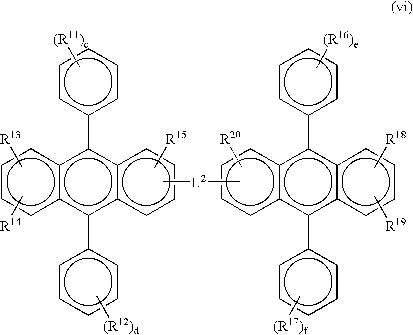

where: R¹¹ to R²⁰ each independently represent a hydrogen atom, an alkyl group, a cycloalkyl group, an aryl group, an alkoxyl group, an aryloxy group, an alkylamino group, an arylamino group, or a heterocyclic group which may be substituted; c, d, e, and f each represent an integer of 1 to 5, and, when any one of c, d, e, and f represents 2 or more, R¹¹'s, R¹²'s, R¹⁶'s or R¹⁷'s may be identical to or different from each other, or R¹¹'s, R¹²'s, R¹⁶'s, or R¹⁷'s may be bonded to each other to form a ring; R¹³ and R¹⁴, or R¹⁸ and R¹⁹ may be bonded to each other to form a ring; and L² represents a single bond, —O—, —S—, —N(R)— (where R represents an alkyl group or an aryl group which may be substituted), an alkylene group, or an arylene group;

a spirofluorene derivative represented by the following general formula (vii):

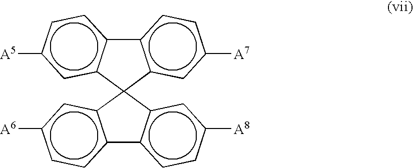

where: A⁵ to A⁸ each independently represent a substituted or unsubstituted biphenylyl group, or a substituted or unsubstituted naphthyl group;

a fused ring-containing compound represented by the following general formula (viii):

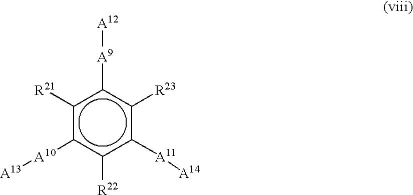

where: A⁹ to A¹⁴ each have the same meaning as that described above; R²¹ to R²³ each independently represent a hydrogen atom, an alkyl group having 1 to 6 carbon atoms, a cycloalkyl group having 3 to 6 carbon atoms, an alkoxyl group having 1 to 6 carbon atoms, an aryloxy group having 5 to 18 carbon atoms, an aralkyloxy group having 7 to 18 carbon atoms, an arylamino group having 5 to 16 carbon atoms, a nitro group, a cyano group, an ester group having 1 to 6 carbon atoms, or a halogen atom; and at least one of A⁹ to A¹⁴ represents a group having three or more fused aromatic rings; and

a fluorene compound represented by the following general formula (ix):

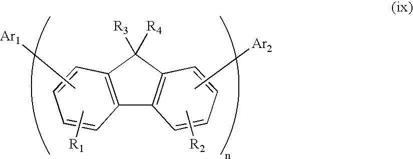

where: R₁ and R₂ each represent a hydrogen atom, a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom; R₁'s or R₂'s bonded to different fluorene groups may be identical to or different from each other, and R₁ and R₂ bonded to the same fluorene group may be identical to or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group; R₃'s or R₄'s bonded to different fluorene groups may be identical to or different from each other, and R₃ and R₄ bonded to the same fluorene group may be identical to or different from each other; Ar₁ and Ar₂ each represent a substituted or unsubstituted fused polycyclic aromatic group having three or more benzene rings in total, or a substituted or unsubstituted fused polycyclic heterocyclic group that has three or more rings each of which is a benzene ring or a heterocyclic ring in total and that is bonded to a fluorene group by carbon, and Ar¹ and Ar₂ may be identical to or different from each other; and n represents an integer of 1 to 10.

Of the above-mentioned host materials, an anthracene derivative is preferable, a monoanthracene derivative is more preferable, and an asymmetric anthracene is particularly preferable.

In addition, a phosphorescent compound can also be used as a light emitting material of a dopant. A compound containing a carbazole ring in a host material is preferable as the phosphorescent compound. The dopant is a compound capable of emitting light from a triplet exciton, and is not particularly limited as long as light is emitted from a triplet exciton, a metal complex containing at least one metal selected from the group consisting of Ir, Ru, Pd, Pt, Os, and Re is preferable, and a porphyrin metal complex or an orthometalated metal complex is preferable.

A host composed of a compound containing a carbazole ring and suitable for phosphorescence is a compound having a function of causing a phosphorescent compound to emit light as a result of the occurrence of energy transfer from the excited state of the host to the phosphorescent compound. The host compound is not particularly limited as long as it is a compound capable of transferring exciton energy to a phosphorescent compound, and can be appropriately selected in accordance with a purpose. The host compound may have, for example, an arbitrary heterocyclic ring in addition to a carbazole ring.

Specific examples of the host compound include carbazole derivatives, triazole derivatives, oxazole derivatives, oxadiazole derivatives, imidazole derivatives, polyarylalkane derivatives, pyrazoline derivatives, pyrazolone derivatives, phenylene diamine derivatives, arylamine derivatives, amino substituted chalcone derivatives, styrylanthracene derivatives, fluorenone derivatives, hydrazone derivatives, stilbene derivatives, silazane derivatives, aromatic tertiary amine compounds, styrylamine compounds, aromatic dimethylidene-based compounds, porphyrin-based compounds, anthraquinodimethane derivatives, anthrone derivatives, diphenylquinone derivatives, thiopyranedioxide derivatives, carbodiimide derivatives, fluorenilidene methane derivatives, distyryl pyrazine derivatives, heterocyclic tetracarboxylic anhydrides such as naphthaleneperylene, phthalocyanine derivatives, various metal complex polysilane-based compounds typified by a metal complex of an 8-quinolinol derivative or a metal complex having metal phthalocyanine, benzooxazole, or benzothiazole as a ligand, poly(N-vinylcarbazole) derivatives, aniline-based copolymers, conductive high molecular weight oligomers such as a thiophene oligomer and polythiophene, polymer compounds such as polythiophene derivatives, polyphenylene derivatives, polyphenylene vinylene derivatives, and polyfluorene derivatives. One of the host materials may be used alone, or two or more of them may be used in combination.

Specific examples thereof include the compounds as described below.

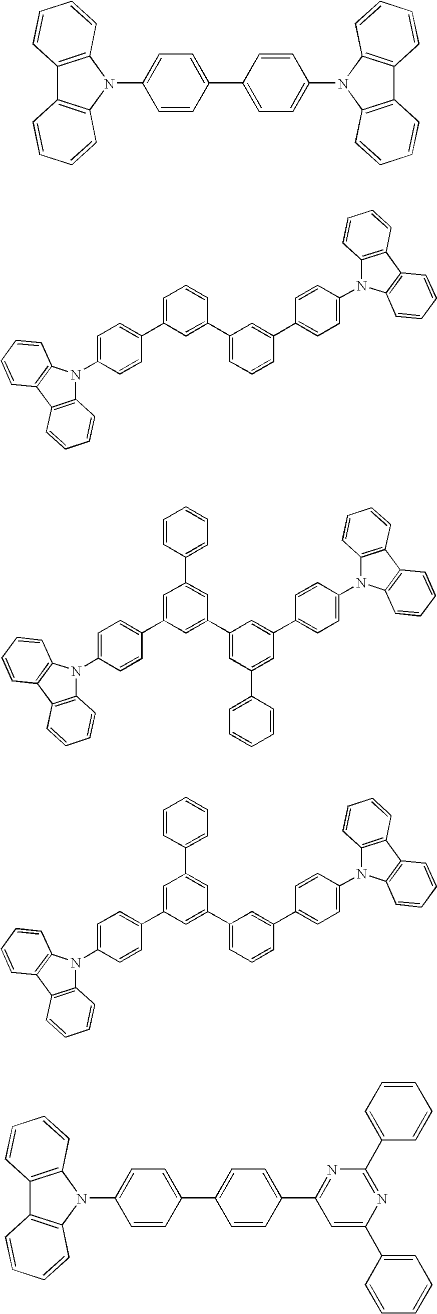

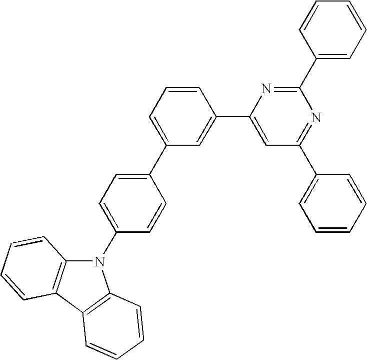

A phosphorescent dopant is a compound capable of emitting light from a triplet exciton. The dopant, which is not particularly limited as long as light is emitted from a triplet exciton, is preferably a metal complex containing at least one metal selected from the group consisting of Ir, Ru, Pd. Pt, Os, and Re, and is preferably a porphyrin metal complex or an orthometalated metal complex. A porphyrin platinum complex is preferable as the porphyrin metal complex. One kind of a phosphorescent compound may be used alone, or two or more kinds of phosphorescent compounds may be used in combination.

Any one of various ligands can be used for forming an orthometalated metal complex. Examples of a preferable ligand include 2-phenyl pyridine derivatives, 7,8-benzoquinoline derivatives, 2-(2-thienyl)pyridine derivatives, 2-(1-naphthyl)pyridine derivatives, and 2-phenyl quinoline derivatives. Each of those derivatives may have a substituent as required. A fluoride of any one of those derivatives, or one obtained by introducing a trifluoromethyl group into any one of those derivatives is a particularly preferable blue-based dopant. The metal complex may further include a ligand other than the above-mentioned ligands such as acetylacetonato or picric acid as an auxiliary ligand.

The content of the phosphorescent dopant in the light emitting layer is not particularly limited, and can be appropriately selected in accordance with a purpose. The content is, for example, 0.1 to 70 mass %, and is preferably 1 to 30 mass %. When the content of the phosphorescent compound is less than 0.1 mass %, the intensity of emitted light is weak, and an effect of the incorporation of the compound is not sufficiently exerted. When the content exceeds 70 mass %, a phenomenon referred to as concentration quenching becomes remarkable, and device performance reduces.

In addition, the light emitting layer may contain a hole transporting material, an electron transporting material, or a polymer binder as required.

Further, the thickness of the light emitting layer is preferably 5 to 50 nm, more preferably 7 to 50 nm, or most preferably 10 to 50 nm. When the thickness is less than 5 nm, it becomes difficult to form the light emitting layer, so the adjustment of chromaticity may be difficult. When the thickness exceeds 50 nm, the driving voltage may increase.

(5) Hole Injecting and Transporting Layer (Hole Transporting Zone)

The hole injecting and transporting layer is a layer which helps injection of holes into the light emitting layer and transports the holes to the light emitting region. The layer exhibits a great mobility of holes and, in general, has an ionization energy as small as 5.6 eV or smaller. For the hole injecting and transporting layer, a material which transports holes to the light emitting layer under an electric field of a smaller strength is preferable. A material which exhibits, for example, a mobility of holes of at least 10-4 cm²/V·sec under application of an electric field of 10⁴ to 10⁶ V/cm is preferable.

When the aromatic amine derivative of the present invention is used in the hole transporting zone, the aromatic amine derivative of the present invention may be used alone or as a mixture with other materials for forming the hole injecting and transporting layer.

The material which can be used for forming the hole injecting and transporting layer as a mixture with the aromatic amine derivative of the present invention is not particularly limited as long as the material has a preferable property described above. The material can be arbitrarily selected from materials which are conventionally used as the charge transporting material of holes in photoconductive materials and known materials which are used for the hole injecting and transporting layer in organic EL devices. In the present invention, the material having a transporting property of holes and being able to be used for the hole transporting zone is referred to as a hole transporting material.

Specific examples include: a triazole derivative (see, for example, U.S. Pat. No. 3,112,197); an oxadiazole derivative (see, for example, U.S. Pat. No. 3,189,447); an imidazole derivative (see, for example, Japanese Examined Patent Publication No. Sho 37-16096); a polyarylalkane derivative (see, for example, U.S. Pat. Nos. 3,615,402, 3,820,989, and 3,542,544, Japanese Examined Patent Publication Nos. Sho 45-555 and 51-10983, Japanese Patent Application Laid-Open Nos. Sho 51-93224, 55-17105, 56-4148, 55-108667, 55-156953, and 56-36656); a pyrazoline derivative and a pyrazolone derivative (see, for example, U.S. Pat. Nos. 3,180,729, and 4,278,746, Japanese Patent Application Laid-Open Nos. Sho 55-88064, 55-88065, 49-105537, 55-51086, 56-80051, 56-88141, 57-45545, 54-112637, and 55-74546); a phenylenediamine derivative (see, for example, U.S. Pat. No.3,615,404, Japanese Examined Patent Publication Nos. Sho S5-1010S, 46-3712, and 47-25336, and Japanese Patent Application Laid-Open No. Sho 54-119925); an arylamine derivative (see, for example, U.S. Pat. Nos. 3,567,450, 3,240,597, 3,658,520, 4,232,103, 4,175,961, and 4,012,376, Japanese Examined Patent Publication Nos. Sho 49-35702, and 39-27577, Japanese Patent Application Laid-Open Nos. Sho 55-144250, 56-119132, 56-22437, and German Patent No. 1,110,518); an amino-substituted chalcone derivative (see, for example, U.S. Pat. No. 3,526,501); an oxazole derivative (those disclosed in U.S. Pat. No. 3,257,203); a styrylanthracene derivative (see, for example, Japanese Patent Application Laid-Open No. Sho 56-46234); a fluorenone derivative (see, for example, Japanese Patent Application Laid-Open No. Sho 54-110837); a hydrazone derivative (see, for example, U.S. Pat. No. 3,717,462, Japanese Patent Application Laid-Open Nos. Sho 54-59143, 55-52063, 55-52064, 55-46760, 57-11350, 57-148749, and 2-311591); a stilbene derivative (see, for example, Japanese Patent Application Laid-Open Nos. Sho 61-210363, 61-228451, 61-14642, 61-72255, 62-47646, 62-36674, 62-10652, 62-30255, 60-93455, 60-94462, 60-174749, and 60-175052); a silazane derivative (U.S. Pat. No. 4,950,950); a polysilane-based copolymer (Japanese Patent Application Laid-Open No. 2-204996); an aniline-based copolymer (Japanese Patent Application Laid-Open No. 2-282263); and a conductive high-molecular oligomer (in particular, a thiophene oligomer).

In addition to the above-mentioned materials which can be used as the material for the hole injecting and transporting layer, a porphyrin compound (those disclosed in, for example, Japanese Patent Application Laid-Open No. Sho 63-295695); an aromatic tertiary amine compound and a styrylamine compound (see, for example, U.S. Pat. No. 4,127,412, Japanese Patent Application Laid-Open Nos. Sho 53-27033, 54-58445, 55-79450, 55-144250, 56-119132, 61-295558, 61-98353, and63-295695) are preferable, and aromatic tertiary amine compounds are particularly preferable.

Further, examples of aromatic tertiaryamine compounds include compounds having two fused aromatic rings in the molecule such as 4,4′-bis(N-(1-naphthyl)-N-phenylamino)-biphenyl (hereinafter referred to as NPD) as disclosed in U.S. Pat. No. 5,061,569, and a compound in which three triphenylamine units are bonded together in a star-burst shape, such as 4,4′,44″-tris(N-(3-methylphenyl)-N-phenylamino)triphenylamine (hereinafter referred to as MTDATA) as disclosed in Japanese Patent Application No. 4-308688.

Further, in addition to the aromatic dimethylidine-based compounds described above as the material for the light emitting layer, inorganic compounds such as Si of the p-type and SiC of the p-type can also be used as the material for the hole injecting and transporting layer.

The hole injecting and transporting layer can be formed by forming a thin layer from the aromatic amine derivative of the present invention in accordance with a known process such as the vacuum vapor deposition process, the spin coating process, the casting process, and the LB process. The thickness of the hole injecting and transporting layer is not particularly limited. In general, the thickness is 5 nm to 5 μm. The hole injecting and transporting layer may be formed of a single layer containing one or more materials described above or may be a laminate formed of hole injecting and transporting layers containing materials different from the materials of the hole injecting and transporting layer described above as long as the aromatic amine derivative of the present invention is incorporated in the hole injecting and transporting zone.

Further, an organic semiconductor layer may be disposed as a layer for helping the injection of holes or electrons into the light emitting layer. As the organic semiconductor layer, a layer having a conductivity of 10⁻¹⁰ S/cm or higher is preferable. As the material for the organic semiconductor layer, oligomers containing thiophene, and conductive oligomers such as oligomers containing arylamine and conductive dendrimers such as dendrimers containing arylamine, which are disclosed in Japanese Patent Application No. 8-193191, can be used.

(6) Electron Injecting and Transporting Layer

Next, the electron injecting and transporting layer is a layer which helps injection of electrons into the light emitting layer, transports the holes to the light emitting region, and exhibits a great mobility of electrons. The adhesion improving layer is an electron injecting layer including a material exhibiting particularly improved adhesion with the cathode.

In addition, it is known that, in an organic EL device, emitted light is reflected by an electrode (cathode in this case), so emitted light directly extracted from an anode and emitted light extracted via the reflection by the electrode interfere with each other. The thickness of an electron transporting layer is appropriately selected from the range of several nanometers to several micrometers in order that the interference effect may be effectively utilized. When the thickness is particularly large, an electron mobility is preferably at least 10⁻⁵cm²/Vs or more upon application of an electric field of 10⁴ to 10⁶ V/cm in order to avoid an increase in voltage.

A metal complex of 8-hydroxyquinoline or of a derivative of 8-hydroxyquinoline, or an oxadiazole derivative is suitable as a material to be used in an electron injecting layer. Specific examples of the metal complex of 8-hydroxyquinoline or of the derivative of 8-hydroxyquinoline that can be used as an electron injecting material include metal chelate oxynoid compounds each containing a chelate of oxine (generally 8-quinolinol or 8-hydroxyquinoline), such as tris(8-quinolinol)aluminum.

On the other hand, examples of the oxadiazole derivative include electron transfer compounds represented by the following general formula:

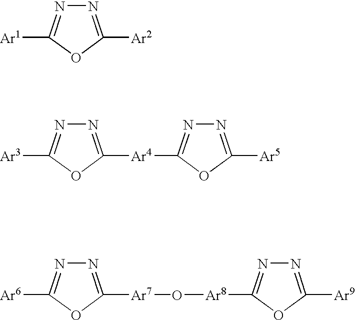

where: Ar¹, Ar², Ar³, Ar⁵, Ar⁶ and Ar⁹ each represent a substituted or unsubstituted aryl group and may represent the same group or different groups; and Ar⁴, Ar⁷ and Ar⁹ each represent a substituted or unsubstituted arylene group and may represent the same group or different groups.

Examples of the aryl group include a phenyl group, a biphenylyl group, an anthryl group, a perylenyl group, and a pyrenyl group. Examples of the arylene group include a phenylene group, a naphthylene group, a biphenylene group, an anthrylene group, a perylenylene group, and a pyrenylene group. Examples of the substituent include alkyl groups each having 1 to 10 carbon atoms, alkoxyl groups each having 1 to 10 carbon atoms, and a cyano group. As the electron transfer compound, compounds which can form thin films are preferable.

Examples of the electron transfer compounds described above include the following.

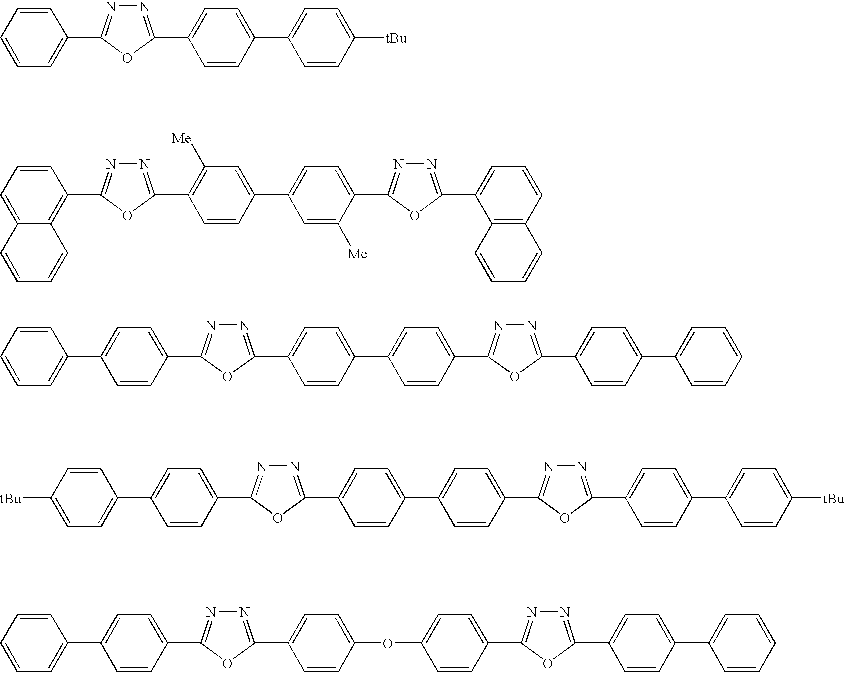

Further, materials represented by the following general formulae (A) to (F) can be used in an electron injecting layer and an electron transporting layer:

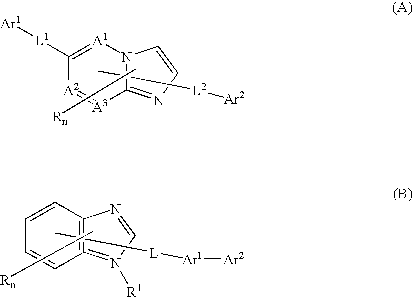

each representing a nitrogen-containing heterocyclic ring derivative, where: A¹ to A³ each independently represent a nitrogen atom or a carbon atom;

Ar¹ represents a substituted or unsubstituted aryl group having 6 to 60 ring carbon atoms, or a substituted or unsubstituted heteroaryl group having 3 to 60 ring carbon atoms, Ar² represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, or a substituted or unsubstituted alkoxy group having 1 to 20 carbon atoms, or a divalent group of any one of them, provided that one of Ar¹ and Ar² represents a substituted or unsubstituted fused ring group having 10 to 60 ring carbon atoms or a substituted or unsubstituted monohetero fused ring group having 3 to 60 ring carbon atoms, or a divalent group of any one of them;

L¹, L², and L each independently represent a single bond, a substituted or unsubstituted arylene group having 6 to 60 ring carbon atoms, a substituted or unsubstituted heteroarylene group having 3 to 60 ring carbon atoms, or a substituted or unsubstituted fluorenylene group; and

R represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, or a substituted or unsubstituted alkoxy group having 1 to 20 carbon atoms, n represents an integer of 0 to 5, and, when n represents 2 or more, a plurality of R's may be identical to or different from each other, and a plurality of R groups adjacent to each other may be bonded to each other to form a carbocyclic aliphatic ring or a carbocyclic aromatic ring;

R¹ represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, or a substituted or unsubstituted alkoxy group having 1 to 20 carbon atoms, or -L-Ar¹—Ar².

HAr-L-Ar¹—Ar² (C)

representing a nitrogen-containing heterocyclic ring derivative, where: HAr represents a nitrogen-containing heterocyclic ring which has 3 to 40 carbon atoms and may have a substituent; L represents a single bond, an arylene group which has 6 to 60 carbon atoms and may have a substituent, a heteroarylene group which has 3 to 60 carbon atoms and may have a substituent, or a fluorenylene group which may have a substituent; Ar¹ represents a divalent aromatic hydrocarbon group which has 6 to 60 carbon atoms and may have a substituent; and Ar² represents an aryl group which has 6 to 60 carbon atoms and may have a substituent, or a heteroaryl group which has 3 to 60 carbon atoms and may have a substituent;

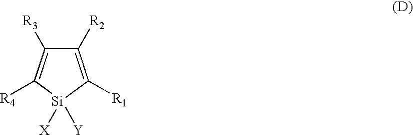

representing a silacyclopentadiene derivative, where: X and Y each independently represent a saturated or unsaturated hydrocarbon group having 1 to 6 carbon atoms, an alkoxy group, an alkenyloxy group, an alkynyloxy group, a hydroxyl group, a substituted or unsubstituted aryl group, or a substituted or unsubstituted heterocycle, or X and Y are bonded to each other to form a structure as a saturated or unsaturated ring; and R₁ to R₄ each independently represent hydrogen, a halogen atom, a substituted or unsubstituted alkyl group having 1 to 6 carbon atoms, an alkoxy group, an aryloxy group, a perfluoroalkyl group, a perfluoroalkoxy group, an amino group, an alkylcarbonyl group, an arylcarbonyl group, an alkoxycarbonyl group, an aryloxycarbonyl group, an azo group, an alkylcarbonyloxy group, an arylcarbonyloxy group, an alkoxycarbonyloxy group, an aryloxycarbonyloxy group, a sulfinyl group, a sulfonyl group, a sulfanyl group, a silyl group, carbamoyl group, an aryl group, a heterocyclic group, an alkenyl group, an alkynyl group, a nitro group, a formyl group, a nitroso group, a formyloxy group, an isocyano group, a cyanate group, an isocyanate group, a thiocyanate group, an isothiocyanate group, or a cyano group, or, when two or more of R₁ to R₄ are adjacent to each other, they form a structure in which a substituted or unsubstituted ring is fused;

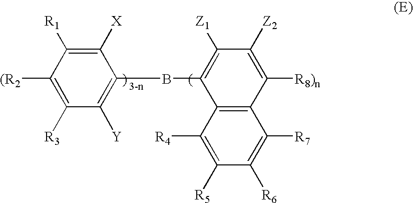

representing a borane derivative, where: R₁ to R₈ and Z₂ each independently represent a hydrogen atom, a saturated or unsaturated hydrocarbon group, an aromatic group, a heterocyclic group, a substituted amino group, a substituted boryl group, an alkoxy group, or an aryloxy group; X, Y, and Z₁ each independently represent a saturated or unsaturated hydrocarbon group, an aromatic group, a heterocyclic group, a substituted amino group, an alkoxy group, or an aryloxy group; substituents of Z₁ and Z₂ may be bonded to each other to form a fused ring; and n represents an integer of 1 to 3, and, when n represents 2 or more, Z₁'s may be different from each other provided that the case where n represents 1, X, Y, and R₂ each represent a methyl group, R₈ represents a hydrogen atom or a substituted boryl group and the case where n represents 3 and Z₁'s each represent a methyl group are excluded; and

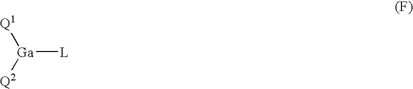

representing a ligand, where: Q¹ and Q² each independently represent a ligand represented by the following general formula (G); and L represents a ligand represented by a halogen atom, a substituted or unsubstituted alkyl group, a substituted or unsubstituted cycloalkyl group, a substituted or unsubstituted aryl group, a substituted or unsubstituted heterocyclic ring group, —OR¹ where R¹ represents a hydrogen atom, a substituted or unsubstituted alkyl group, a substituted or unsubstituted cycloalkyl group, a substituted or unsubstituted aryl group, or a substituted or unsubstituted heterocyclic ring group, or a ligand represented by —O—Ga-Q³(Q⁴) where Q³ and Q⁴ are identical to Q¹ and Q², respectively:

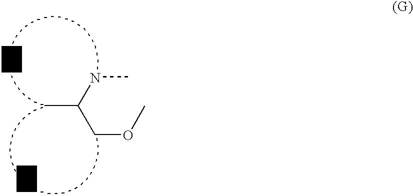

where: rings A¹ and A² are six-membered aryl ring structures which are fused with each other and each of which may have a substituent.

The metal complex behaves strongly as an n-type semiconductor, and has a large electron injecting ability. Further, generation energy upon formation of the complex is low. As a result, the metal and the ligand of the formed metal complex are bonded to each other so strongly that the fluorescent quantum efficiency of the complex as a light emitting material improves.

Specific examples of a substituent in the rings A¹ and A² which each form a ligand of the general formula (G) include: a halogen atom such as chlorine, bromine, iodine, or fluorine; a substituted or unsubstituted alkyl group such as a methyl group, an ethyl group, a propyl group, a butyl group, an s-butyl group, a t-butyl group, a pentyl group, a hexyl group, a heptyl group, an octyl group, a stearyl group, or trichloromethyl group; a substituted or unsubstituted aryl group such as a phenyl group, a naphthyl group, a 3-methylphenyl group, a 3-methoxyphenyl group, a 3-fluorophenyl group, a 3-trichloromethylphenyl group, a 3-trifluoromethylphenyl group, or a 3-nitrophenyl group; a substituted or unsubstituted alkoxy group such as a methoxy group, an n-butoxy group, a t-butoxy group, a trichloromethoxy group, a trifluoroethoxy group, a pentafluoropropoxy group, a 2,2,3,3-tetrafluoropropoxy group, an 1,1,1,3,3,3-hexafluoro-2-propoxy group, or a 6-(perfluoroethyl)hexyloxy group; a substituted or unsubstituted aryloxy group such as a phenoxy group, a p-nitrophenoxy group, a p-t-butylphenoxy group, a 3-fluorophenoxy group, a pentafluorophenyl group, or a 3-trifluoromethylphenoxy group; a substituted or unsubstituted alkylthio group such as a methylthio group, an ethylthio group, a t-butylthio group, a hexylthio group, an octylthio group, or a trifluoromethylthio group; a substituted or unsubstituted arylthio group such as a phenylthio group, a p-nitrophenylthio group, a p-t-butylphenylthio group, a 3-fluorophenylthio group, a pentafluorophenylthio group, or a 3-trifluoromethylphenylthio group; a mono-substituted or di-substituted amino group such as a cyano group, a nitro group, an amino group, a methylamino group, a diethylamino group, an ethylamino group, a diethylamino group, a dipropylamino group, a dibutylamino group, or a diphenylamino group; an acylamino group such as a bis(acetoxymethyl)amino group, a bis(acetoxyethyl)amino group, a bis (acetoxypropyl) amino group, or abis (acetoxybutyl) amino group; a carbamoyl group such as a hydroxyl group, a siloxy group, an acyl group, a methylcarbamoyl group, a dimethylcarbamoyl group, an ethylcarbamoyl group, a diethylcarbamoyl group, a propylcarbamoyl group, a butylcarbamoyl group, or a phenylcarbamoyl group; a cycloalkyl group such as a carboxylic acid group, a sulfonic acid group, an imide group, a cyclopentane group, or a cyclohexyl group; an aryl group such as a phenyl group, a naphthyl group, a biphenylyl group, an anthryl group, a phenanthryl group, a fluorenyl group, or a pyrenyl group; and a heterocyclic group such as a pyridinyl group, a pyrazinyl group, a pyrimidinyl group, a pyridazinyl group, a triazinyl group, an indolinyl group, a quinolinyl group, an acridinyl group, a pyrrolidinyl group, a dioxanyl group, a piperidinyl group, a morpholidinyl group, a piperazinyl group, a triathinyl group, a carbazolyl group, a furanyl group, a thiophenyl group, anoxazolyl group, an oxadiazolyl group, a benzoxazolyl group, a thiazolyl group, a thiadiazolyl group, a benzothiazolyl group, a triazolyl group, an imidazolyl group, a benzoimidazolyl group, or a puranyl group. In addition, the above-mentioned substituents may be bound to each other to further form a six-membered aryl ring or a heterocycle.

A preferable embodiment of the organic EL device of the present invention includes an element including a reducing dopant in the region of electron transport or in the interfacial region of the cathode and the organic layer. The reducing dopant is defined as a substance which can reduce a compound having the electron transporting property. Various substances can be used as the reducing dopant as long as the substances have a uniform reductive property. For example, at least one substance selected from the group consisting of alkali metals, alkaline earth metals, rare earth metals, alkali metal oxides, alkali metal halides, alkaline earth metal oxides, alkaline earth metal halides, rare earth metal oxides, rare earthmetal halides, organic complexes of alkali metals, organic complexes of alkaline earth metals, and organic complexes of rare earth metals can be preferably used.

More specifically, preferable examples of the reducing dopant include at least one alkali metal selected from the group consisting of Na (the work function: 2.36 eV), K (the work function: 2.28 eV), Rb (the work function: 2.16 eV), and Cs (the work function: 1.95 eV) and at least one alkaline earth metal selected from the group consisting of Ca (the work function: 2.9 eV), Sr (the work function: 2.0 to 2.5 eV), and Ba (the work function: 2.52 eV). Particularly preferred are substances having a work function of 2.9 eV or smaller. Of those, at least one alkali metal selected from the group consisting of K, Rb, and Cs is more preferable, Rb and Cs are still more preferable, and Cs is most preferable as the reducing dopant. In particular, those alkali metals have great reducing ability, and the luminance of the emitted light and the lifetime of the organic EL device can be increased by addition of a relatively small amount of the alkali metal into the electron injecting zone. As the reducing dopant having a work function of 2.9 eV or smaller, combinations of two or more alkali metals thereof are also preferable. Combinations having Cs such as the combinations of Cs and Na, Cs and K, Cs and Rb, and Cs, Na, and K are particularly preferable. The reducing ability can be efficiently exhibited by the combination having Cs. The luminance of emitted light and the lifetime of the organic EL device can be increased by adding the combination having Cs into the electron injecting zone.

The present invention may further include an electron injecting layer which is composed of an insulating material or a semiconductor and disposed between the cathode and the organic layer. At this time, the electron injecting property can be improved by preventing a leak of electric current effectively. As the insulating material, at least one metal compound selected from the group consisting of alkali metal chalcogenides, alkaline earth metal chalcogenides, alkali metal halides, and alkaline earth metal halides is preferable. It is preferable that the electron injecting layer be composed of the above-mentioned substance such as the alkali metal chalcogenide since the electron injecting property can be further improved. To be specific, preferable examples of the alkali metal chalcogenide include Li₂O, K₂O, Na₂S, Na₂Se, and Na₂O. Preferable examples of the alkaline earth metal chalcogenide include CaO, BaO, SrO, BeO, BaS, and CaSe. Preferable examples of the alkali metal halide include LiF, NaF, KF, LiCl, KCl, and NaCl. Preferable examples of the alkaline earth metal halide include fluorides such as CaF₂, BaF₂, SrF₂, MgF₂, and BeF₂ and halides other than the fluorides.

Examples of the semiconductor composing the electron transporting layer include oxides, nitrides, and oxide nitrides of at least one element selected from Ba, Ca, Sr, Yb, Al, Ga, In, Li, Na, Cd, Mg, Si, Ta, Sb, and Zn used alone or in combination of two or more. It is preferable that the inorganic compound composing the electron transporting layer form a crystallite or amorphous insulating thin film. When the electron transporting layer is composed of the insulating thin film described above, a more uniform thin film can be formed, and defects of pixels such as dark spots can be decreased. Examples of the inorganic compound include alkali metal chalcogenides, alkaline earth metal chalcogenides, alkali metal halides, and alkaline earth metal halides which are described above.

(7) Cathode

For the cathode, a material such as a metal, an alloy, an electroconductive compound, or a mixture of those materials which has a small work function (4 eV or smaller) is used as an electrode material because the cathode is used for injecting electrons to the electron injecting and transporting layer or the light emitting layer. Specific examples of the electrode material include sodium, sodium-potassium alloys, magnesium, lithium, magnesium-silver alloys, aluminum/aluminum oxide, aluminum-lithium alloys, indium, and rare earth metals.

The cathode can be prepared by forming a thin film of the electrode material described above in accordance with a process such as the vapor deposition process or the sputtering process.

When the light emitted from the light emitting layer is obtained through the cathode, it is preferable that the cathode have a transmittance of the emitted light higher than 10%.

It is also preferable that the sheet resistivity of the cathode be several hundred Ω/□ or smaller. The thickness of the cathode is, in general, selected in the range of 10 nm to 1 μm and preferably in the range of 50 to 200 nm.

(8) Insulating Layer

Defects in pixels tend to be formed in organic EL device due to leak and short circuit since an electric field is applied to ultra-thin films. In order to prevent the formation of the defects, a layer of a thin film having an insulating property may be inserted between the pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. Mixtures and laminates of the above-mentioned compounds may also be used.

(9) Method of Producing the Organic EL Device

In order to prepare the organic EL device of the present invention, the anode and the light emitting layer, and, where necessary, the hole injecting and transporting layer and the electron injecting and transporting layer are formed in accordance with the illustrated process using the illustrated materials, and the cathode is formed in the last step. The organic EL device may also be prepared by forming the above-mentioned layers in the order reverse to the order described above, i.e., the cathode being formed in the first step and the anode in the last step.

Hereinafter, an embodiment of the process for preparing an organic EL device having a construction in which an anode, a hole injecting layer, a light emitting layer, an electron injecting layer, and a cathode are disposed successively on a light-transmissive substrate will be described.

On a suitable light-transmissive substrate, a thin film made of a material for the anode is formed in accordance with the vapor deposition process or the sputtering process so that the thickness of the formed thin film is 1 μm or smaller and preferably in the range of 10 to 200 nm. The formed thin film is used as the anode. Then, a hole injecting layer is formed on the anode. The hole injecting layer can be formed in accordance with the vacuum vapor deposition process, the spin coating process, the casting process, or the LB process, as described above. The vacuum vapor deposition process is preferable since a uniform film can be easily obtained and the possibility of formation of pin holes is small. When the hole injecting layer is formed in accordance with the vacuum vapor deposition process, in general, it is preferable that the conditions be suitably selected in the following ranges: the temperature of the source of the deposition: 50 to 450° C.; the vacuum: 10⁻⁷ to 10⁻³ Torr; the rate of deposition: 0.01 to 50 nm/second; the temperature of the substrate: −50 to 300° C.; and the thickness of the film: 5 nm to 5 μm although the conditions of the vacuum vapor deposition are different depending on the compound to be used (i.e., material for the hole injecting layer) and the crystal structure and the recombination structure of the target hole injecting layer.

Then, the light emitting layer is formed on the hole injecting layer formed above. A thin film of the organic light emitting material can be formed by using a desired organic light emitting material in accordance with a process such as the vacuum vapor deposition process, the sputtering process, the spin coating process, or the casting process, and the formed thin film is used as the light emitting layer. The vacuum vapor deposition process is preferable since a uniform film can be easily obtained and the possibility of formation of pin holes is small. When the light emitting layer is formed in accordance with the vacuum vapor deposition process, in general, the conditions of the vacuum vapor deposition process can be selected in the same ranges as the conditions described for the vacuum vapor deposition of the hole injecting layer, although the conditions are different depending on the compound to be used.

Next, an electron injecting layer is formed on the light emitting layer formed above. Similarly to the hole injecting layer and the light emitting layer, it is preferable that the electron injecting layer be formed in accordance with the vacuum vapor deposition process since a uniform film must be obtained. The conditions of the vacuum vapor deposition can be selected in the same ranges as the condition described for the vacuum vapor deposition of the hole injecting layer and the light emitting layer.

When the vapor deposition process is used, the aromatic amine derivative of the present invention can be deposited by vapor in combination with other materials, although the situation may be different depending on which layer in the light emitting zone or in the hole transporting zone includes the compound. When the spin coating process is used, the compound can be incorporated into the formed layer by using a mixture of the compound with other materials.

A cathode is laminated in the last step, and an organic EL device can be obtained.

The cathode is formed of a metal and can be formed in accordance with the vacuum vapor deposition process or the sputtering process. It is preferable that the vacuum vapor deposition process be used in order to prevent formation of damages on the lower organic layers during the formation of the film.

In the above-mentioned preparation of the organic EL device, it is preferable that the above-mentioned layers from the anode to the cathode be formed successively while the preparation system is kept in a vacuum after being evacuated once.

The method of forming the layers in the organic EL device of the present invention is not particularly limited. A conventionally known process such as the vacuum vapor deposition process or the spin coating process can be used. The organic thin film layer which is used in the organic EL device of the present invention and includes the compound represented by general formula (1) described above can be formed in accordance with a known process such as the vacuum vapor deposition process or the molecular beam epitaxy process (MBE process) or, using a solution prepared by dissolving the compounds into a solvent, in accordance with a coating process such as the dipping process, the spin coating process, the casting process, the bar coating process, or the roll coating process.

The thickness of each layer in the organic thin film layer in the organic EL device of the present invention is not particularly limited. In general, an excessively thin layer tends to have defects such as pin holes, whereas an excessively thick layer requires a high applied voltage to decrease the efficiency. Therefore, a thickness in the range of several nanometers to 1 μm is preferable.

The organic EL device which can be prepared as described above emits light when a direct voltage of 5 to 40 V is applied in the condition that the polarity of the anode is positive (+) and the polarity of the cathode is negative (−). When the polarity is reversed, no electric current is observed and no light is emitted at all. When an alternating voltage is applied to the organic EL device, the uniform light emission is observed only in the condition that the polarity of the anode is positive and the polarity of the cathode is negative. When an alternating voltage is applied to the organic EL device, any type of wave shape can be used.

### EXAMPLES

Hereinafter, the present invention is described in more detail on the basis of Synthesis Examples and Examples.

The structural formulae of Intermediates 1 to 4 to be produced in Synthesis Examples 1 to 4 are as shown below.

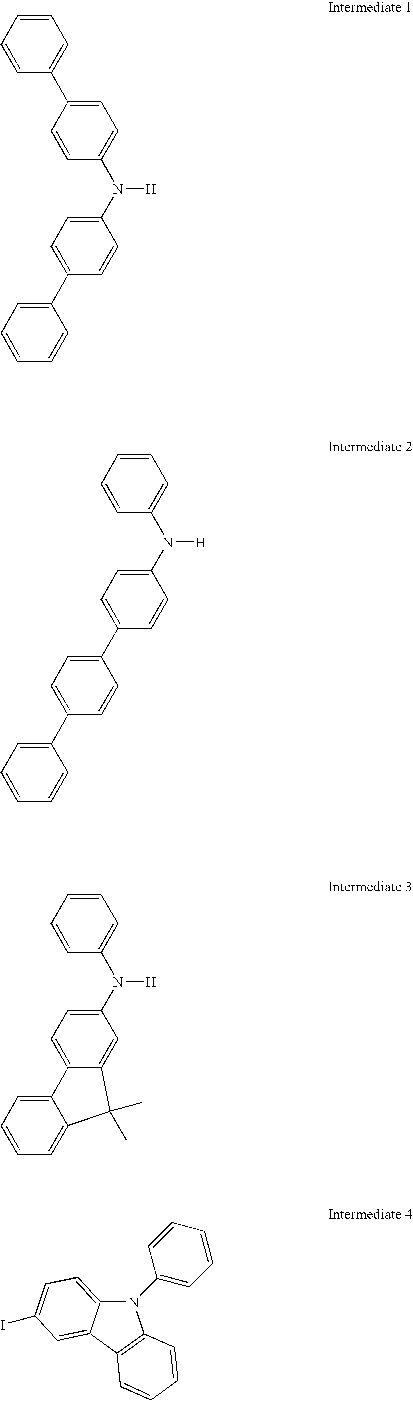

### Synthesis Example 1 (Synthesis of Intermediate 1)

20.0 g of 4-bromobiphenyl (manufactured by Tokyo Chemical Industry Co., Ltd.), 8.64 g of t-butoxysodium (manufactured by Wako Pure Chemical Industries, Ltd.), and 84 mg of palladium acetate (manufactured by Wako Pure Chemical Industries, Ltd.) were loaded into a 200-mL three-necked flask. Further, a stirrer was loaded into the flask, and a rubber cap was set on each of both sides of the flask. A breathing tube for reflux was set at the central port of the rubber cap, and a three-way cock and a balloon in which an argon gas was sealed were set on the tube so that the inside of the system was replaced with the argon gas in the balloon three times by using a vacuum pump.

Next, 120 mL of dehydrated toluene (manufactured by Hiroshima Wako), 4.08 mL of benzylamine (manufactured by Tokyo Chemical Industry Co., Ltd.), 338 μL of tris-t-butylphosphine (manufactured by SIGMA-ALDRICH Corp., 2.22-mol/L toluene solution) were added to the flask with a syringe through a rubber septum, and the mixture was stirred for 5 minutes at room temperature. Next, the flask was set in an oil bath, and the temperature of the solution in the flask was gradually increased to 120° C. while the solution was stirred. 7 hours after that, the flask was removed from the oil bath so that the reaction was terminated, and then the resultant was left to stand under an argon atmosphere for 12 hours. The reaction solution was transferred to a separating funnel, and 600 mL of dichloromethane were added to dissolve the precipitate. The resultant was washed with 120 mL of a saturated salt solution, and then the organic layer was dried with anhydrous potassium carbonate. The solvent of the organic layer obtained by separating potassium carbonate by filtration was removed by distillation. Then, 400 mL of toluene and 80 mL of ethanol were added to the resultant residue, and the mixture was heated to 80° C. by attaching a drying tube to the flask, whereby the residue was completely dissolved. After that, the resultant was left to stand for 12 hours so as to be slowly cooled to room temperature for recrystallization. The precipitated crystal was separated by filtration and dried in a vacuum at 60° C., whereby 13.5 g of N,N-di-(4-biphenylyl)-benzylamine were obtained. 1.35 g of N,N-di-(4-biphenylyl)-benzylamine and 135 mg of palladium-activated carbon (manufactured by Hiroshima Wako, palladium content 10 wt %) were loaded into a 300-mL one-necked flask, and 100 mL of chloroform and 20 mL of ethanol were added to dissolve the mixture. Next, a stirrer was loaded into the flask, and then a three-way cock mounted with a balloon filled with 2L of a hydrogen gas was attached to the flask so that the inside of the flask system was replaced with the hydrogen gas ten times by using a vacuum pump. The balloon was newly filled with the hydrogen gas in an amount corresponding to the consumed amount so that the volume of the hydrogen gas was returned to 2 L. After that, the solution was vigorously stirred at room temperature for 30 hours. After the stirring, 100 mL of dichloromethane were added to the solution, and the catalyst was separated by filtration. Next, the resultant solution was transferred to a separating funnel and washed with 50 mL of a saturated aqueous solution of sodium hydrogen carbonate. After that, the organic layer was separated and dried with anhydrous potassium carbonate. After the resultant had been filtrated, the solvent was removed by distillation, and 50 mL of toluene were added to the resultant residue for recrystallization. The precipitated crystal was separated by filtration and dried in a vacuum at 50° C., whereby 0.99 g of di-4-biphenylylamine (Intermediate 1) was obtained. The resultant compound was identified as Intermediate 1 by FD-MS analysis.

### Synthesis Example 2 (Synthesis of Intermediate 2)

In a stream of argon, 5.5 g of aniline, 15.7 g of 4-bromo-p-terphenyl, 6.8 g of t-butoxysodium (manufactured by Hiroshima Wako), 0.46 g of tris(dibenzylideneacetone)dipalladium(0) (manufactured by SIGMA-ALDRICH Corp.), and 300 mL of dehydrated toluene were loaded into a flask, and the mixture was subjected to a reaction at 80° C. for 8 hours.

After the resultant had been cooled, 500 mL of water were added to the resultant, and the mixture was subjected to cerite filtration. The filtrate was extracted with toluene and dried with anhydrous magnesium sulfate. The dried product was concentrated under reduced pressure, and the resultant coarse product was subjected to column purification, recrystallized with toluene, and taken by filtration. After that, the resultant was dried, whereby 10.8 g of a pale yellow powder were obtained. The powder was identified as Intermediate 2 by FD-MS analysis.

### Synthesis Example 3 (Synthesis of Intermediate 3)

7.3 g of a white powder were obtained by performing a reaction in the same manner as in the synthesis of Intermediate 2 except that 4-bromo-9,9-dimethylfluorene was used instead of 4-bromo-p-terphenyl. The powder was identified as Intermediate 3 by FD-MS analysis.

### Synthesis Example 4 (Synthesis of Intermediate 4)

17.7 g of 9-phenylcarbazole, 6.03 g of potassium iodide, 7.78 g of potassium iodate, 5.90 mL of sulfuric acid, and ethanol were loaded into a flask, and the mixture was subjected to a reaction at 75° C. for 2 hours.

After the resultant had been cooled, distilled water and ethyl acetate were added to the resultant for separation and extraction. After that, the organic layer was washed with baking soda water and distilled water, and was concentrated. The resultant coarse product was purified by silica gel chromatography (toluene), and the resultant solid was dried under reduced pressure, whereby 21.8 g of a white solid were obtained.

In a stream of argon, dehydrated toluene and dehydrated ether were added to 13.1 g of the above resultant white solid, and the mixture was cooled to −45° C. 25 mL of a solution (1.58 M) of n-butyllithium in hexane were dropped to the mixture, and the temperature of the whole was increased to −5° C. over 1 hour while the whole was stirred. After the resultant had been cooled to −45° C. again, 25 mL of a boric acid triisopropyl ester were slowly dropped to the resultant, and the mixture was subjected to a reaction for 2 hours.

After the temperature of the resultant had been returned to room temperature, a 10% dilute hydrochloric acid solution was added to the resultant, and the mixture was stirred so that an organic layer was extracted. After having been washed with a saturated salt solution, the organic layer was dried with anhydrous magnesium sulfate, separated by filtration, and concentrated. The resultant solid was purified by silica gel chromatography (toluene), and the resultant solid was washed with n-hexane and dried under reduced pressure, whereby 7.10 g of a solid were obtained. The solid was identified as Intermediate 4 by FD-MS analysis.

The structural formulae of Compounds H1 to H12 to be produced in Examples-of-Synthesis 1 to 12 each serving as the aromatic amine derivative of the present invention are as shown below.

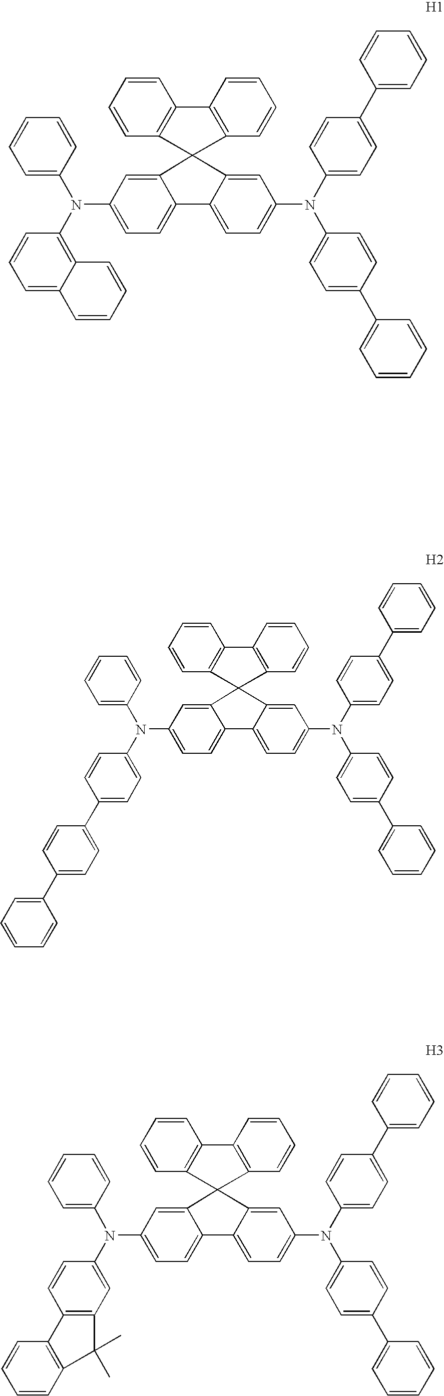

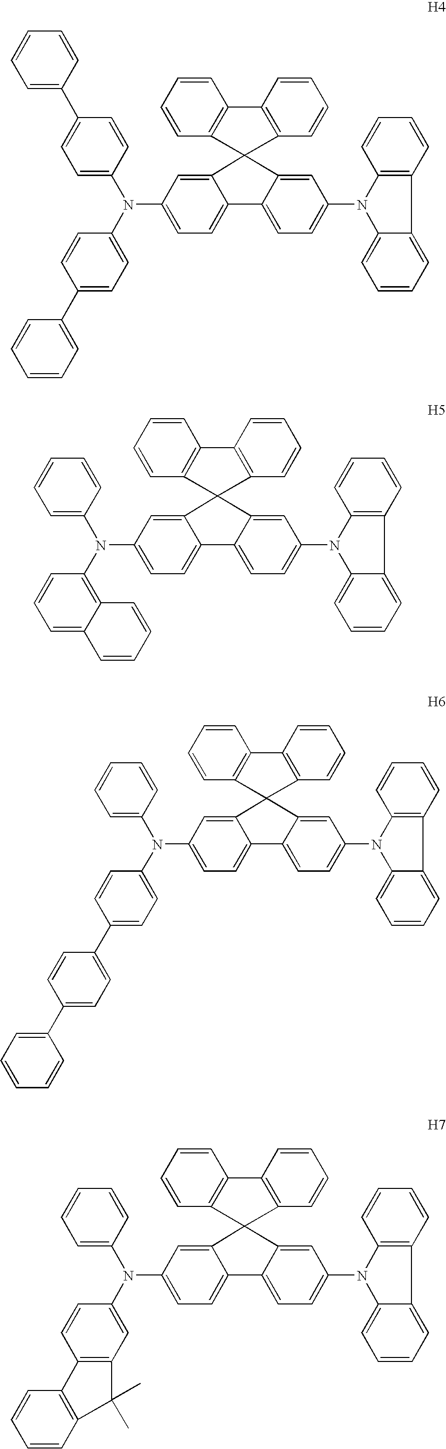

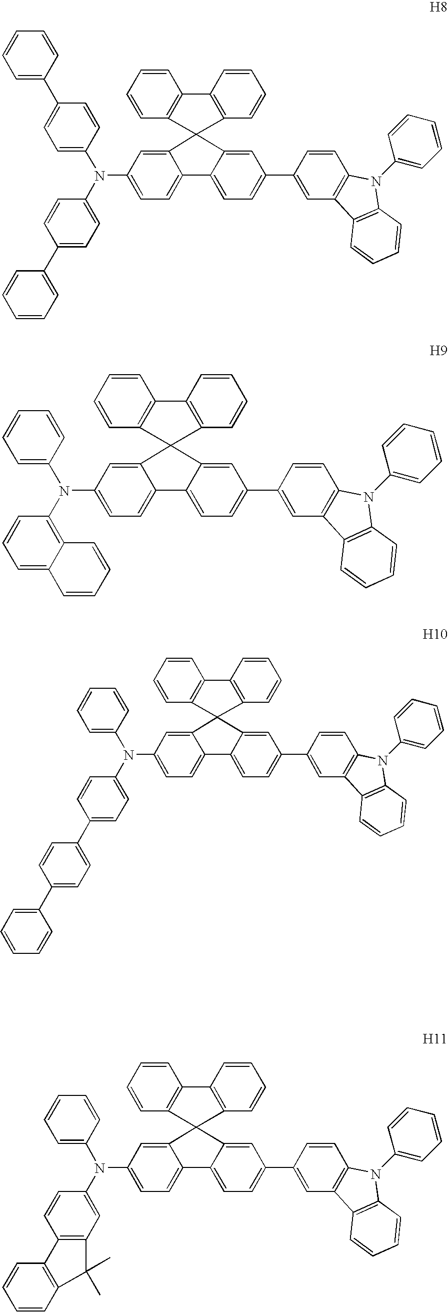

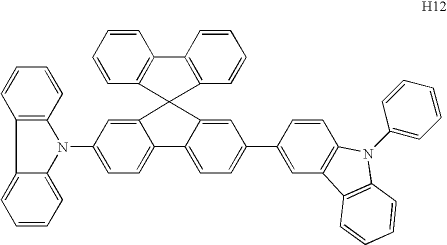

### Example-of-Synthesis 1 (Synthesis of Compound H1)

The following first reaction was performed: in a stream of argon, 6.4 g of Intermediate 1, 9.5 g of 2,2′-dibromo-9,9′-spirobisfluorene, 231 mg of Pd₂(dba)₃, 325 mg of P(t-Bu)₃, 2.9 g of t-butoxysodium, and toluene were loaded into a flask, and the mixture was subjected to a reaction at 80° C. for 4 hours. After the resultant had been cooled, toluene was added to the resultant, and the mixture was subjected to cerite filtration. After that, the filtrate was concentrated. The concentrated product was purified by silica gel chromatography (hexane:dichloromethane =6:1), and the resultant solid was washed with n-hexane and dried under reduced pressure, whereby 2.1 g of a white solid were obtained.

The following second reaction was performed: the above resultant compound and 1-naphthylphenylamine were subjected to a reaction in the same manner as in the first reaction. As a result, 1.3 g of a white solid were obtained. The solid was identified as Compound H1 by FD-MS analysis.

### Example-of-Synthesis 2 (Synthesis of Compound H2)

0.9 g of a whitish yellow solid was obtained by performing the second reaction in the same manner as in Example-of-Synthesis 1 except that Intermediate 2 was used instead of 1-naphthylphenylamine. The solid was identified as Compound H2 by FD-MS analysis.

### Example-of-Synthesis 3 (Synthesis of Compound H3)

1.0 g of a whitish yellow solid was obtained by performing the second reaction in the same manner as in Example-of-Synthesis 1 except that Intermediate 3 was used instead of 1-naphthylphenylamine. The solid was identified as Compound H3 by FD-MS analysis.

### Example-of-Synthesis 4 (Synthesis of Compound H4)

The following first reaction was performed: in a stream of argon, 3.2 g of carbazole, 9.5 g of 2,2′-dibromo-9,9′-spirobisfluorene, 231 mg of Pd₂(dba)₃, 325 mg of P(t-Bu)₃, 2.9 g of t-butoxysodium, and toluene were loaded into a flask, and the mixture was subjected to a reaction at 80° C. for 4 hours. After the resultant had been cooled, toluene was added to the resultant, and the mixture was subjected to cerite filtration. After that, the filtrate was concentrated. The concentrated product was purified by silica gel chromatography (hexane:dichloromethane =6:1), and the resultant solid was washed with n-hexane and dried under reduced pressure, whereby 1.1 g of a white solid were obtained.

The following second reaction was performed: the above resultant compound and Intermediate 1 were subjected to a reaction in the same manner as in the first reaction. As a result, 0.7 g of a white solid were obtained. The solid was identified as Compound H4 by FD-MS analysis.

### Example-of-Synthesis 5 (Synthesis of Compound H5)

0.9 g of a white solid was obtained by performing the second reaction in the same manner as in Example-of-Synthesis 4 except that 1-naphthylphenylamine was used instead of Intermediate 1. The solid was identified as Compound H5 by FD-MS analysis.

### Example-of-Synthesis 6 (Synthesis of Compound H6)

0.6 g of a white solid was obtained by performing the second reaction in the same manner as in Example-of-Synthesis 4 except that Intermediate 2 was used instead of Intermediate 1. The solid was identified as Compound H6 by FD-MS analysis.

### Example-of-Synthesis 7 (Synthesis of Compound H7)

0.9 g of a white solid was obtained by performing the reactions in the same manner as in Example-of-Synthesis 4 except that carbazole was used instead of Intermediate 1 in the first reaction, and Intermediate 3 was used instead of 1-naphthylphenylamine in the second reaction. The solid was identified as Compound H7 by FD-MS analysis.

### Example-of-Synthesis 8 (Synthesis of Compound H8)

The following first reaction was performed: 22.1 g of Intermediate 4, 23.7 g of 2,2′-dibromo-9,9′-spirobisfluorene, 1.38 g of tetrakis(triphenylphosphine)palladium (Pd(PPh₃)₄), 21.9 g of sodium carbonate, clean water, and dimethoxyethane were loaded into a flask, and the mixture was subjected to a reaction under reflux for 8 hours.

After having been cooled, the reaction solution was filtrated. The residue after the filtration was extracted with acetone, and the separated water layer was extracted with dichloromethane. The collected filtrate was separated by adding acetone and dichloromethane. The residue after the filtration was extracted with acetone, and the separated water layer was extracted with dichloromethane. The collected organic layer was washed with clean water and concentrated, and the resultant coarse product was purified by silica gel chromatography (hexane:dichloromethane=9:1). The resultant solid was recrystallized with toluene and methanol, and was dried under reduced pressure, whereby 4.18 g of a white solid were obtained.

The same second reaction as that of Example-of-Synthesis 4 was performed, whereby 3.2 g of a white solid were obtained. The solid was identified as Compound H8 by FD-MS analysis.

### Example-of-Synthesis 9 (Synthesis of Compound H9)

2.7 g of a white solid was obtained by performing the second reaction in the same manner as in Example-of-Synthesis 8 except that 1-naphthylphenylamine was used instead of Intermediate 1. The solid was identified as Compound H9 by FD-MS analysis.

### Example-of-Synthesis 10 (Synthesis of Compound H10)

2.3 g of a white solid was obtained by performing the second reaction in the same manner as in Example-of-Synthesis 8 except that Intermediate 2 was used instead of Intermediate 1. The solid was identified as Compound H10 by FD-MS analysis.

### Example-of-Synthesis 11 (Synthesis of Compound H11)

3.3 g of a white solid was obtained by performing the second reaction in the same manner as in Example-of-Synthesis 8 except that Intermediate 3 was used instead of Intermediate 1. The solid was identified as Compound H11 by FD-MS analysis.

### Example-of-Synthesis 12 (Synthesis of Compound H12)

2.8 g of a white solid was obtained by performing the second reaction in the same manner as in Example-of-Synthesis 8 except that carbazole was used instead of Intermediate 1. The solid was identified as Compound H12 by FD-MS analysis.

### Example 1 (Production of Organic EL Device)

A glass substrate with an ITO transparent electrode measuring 25 mm wide by 75 mm long by 1.1 mm thick (manufactured by GEOMATEC Co., Ltd.) was subjected to ultrasonic cleaning in isopropyl alcohol for 5 minutes. After that, the substrate was subjected to UW ozone cleaning for 30 minutes.

The glass substrate with the transparent electrode line after the washing was mounted on a substrate holder of a vacuum deposition device. First, Compound H1 described above was formed into a film having a thickness of 80 nm on the surface on the side where the transparent electrode line was formed to cover the transparent electrode. The H1 film functions as a hole injecting layer and a hole transporting layer. Further, Compound EM1 to be described below was deposited from the vapor and formed into a film having a thickness of 40 nm. Simultaneously with this formation, Amine Compound D1 having a styryl group to be described below, as a light emitting molecule, was deposited from the vapor in such a manner that a weight ratio between Compound EM1 and Amine Compound D1 would be 40:2. The film functions as a light emitting layer.

Alq to be described below was formed into a film having a thickness of 10 nm on the resultant film. The film functions as an electron injecting layer. After that, Li serving as a reducing dopant (Li source: manufactured by SAES Getters) and Alq were subjected to co-deposition. Thus, an Alq:Li film (having a thickness of 10 nm) was formed as an electron injecting layer (cathode). Metal Al was deposited from the vapor onto the Alq:Li film to form a metal cathode. Thus, an organic EL device was formed.

In addition, the current efficiency of the resultant organic EL device was measured, and the luminescent color of the device was observed. A current efficiency of 10 mA/cm² was calculated by measuring a luminance by using a CS1000 manufactured by Minolta. Further, the half lifetime of light emission in DC constant current driving at an initial luminance of 5,000 cd/m² and room temperature was measured. Table 1 shows the results.

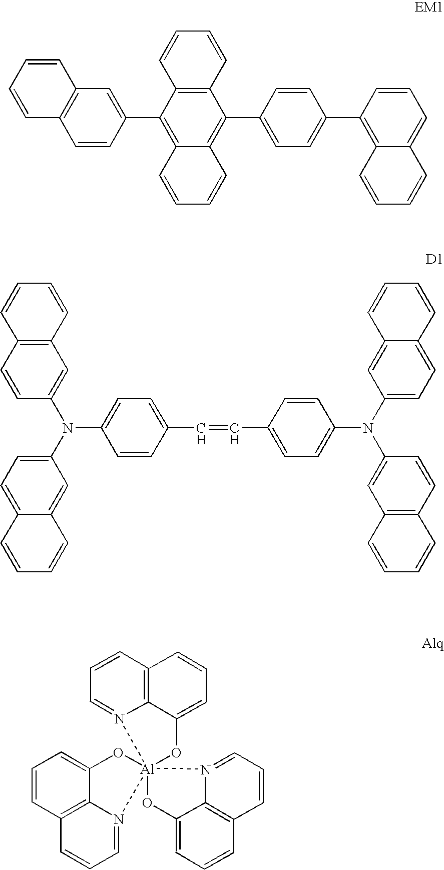

### Examples 2 to 12 (Production of Organic EL Device)

An experiment and measurement were each performed in the same manner as in Example 1 except that a compound described in Table 1 was used instead of Compound H1 as a hole transporting material. Table 1 shows the results.

### Comparative Examples 1 and 2

An experiment and measurement were each performed in the same manner as in Example 1 except that Comparative Compound 1 or Comparative Compound 2 was used instead of Compound H1 as a hole transporting material. Table 1 shows the results.

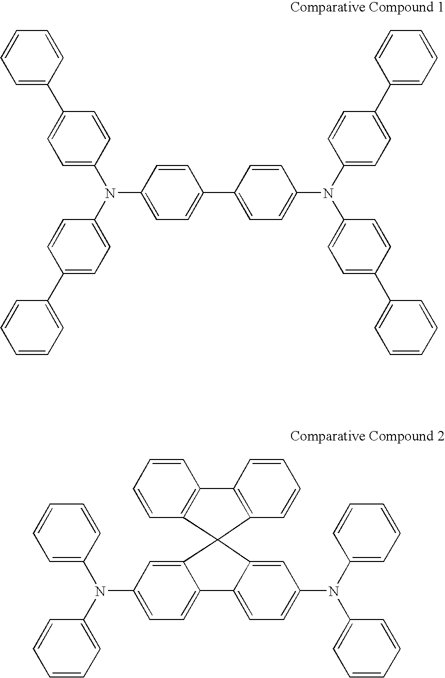

### Example 13 (Production of Organic EL Device)

An experiment and measurement were each performed in the same manner as in Example 1 except that the following Arylamine Compound D2 was used instead of the Amine Compound D1 having a styryl group. Table 1 shows the results.

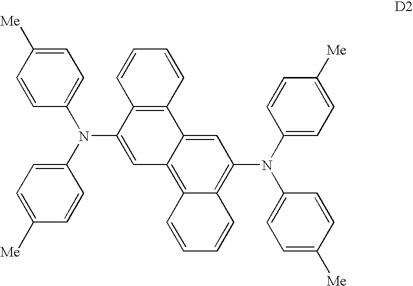

### Comparative Example 3

An experiment and measurement were each performed in the same manner as in Example 13 except that the above-mentioned Comparative Compound 1 was used instead of Compound H1 as a hole transporting material. Table 1 shows the results.

### Example 14 (Production of Organic EL Device)

An experiment and measurement were each performed in the same manner as in Example 4 except that the following Arylamine Compound was used instead of the Amine Compound Di having a styryl group. Table 1 shows the results.

TABLE 1

| | | Current | | Half |
|---|---|---|---|---|
| | Hole transporting | efficiency | Luminescent | lifetime |
| | material | (cd/A) | color | (h) |
| Example 1 | H1 | 4.9 | Blue | 350 |
| Example 2 | H2 | 4.8 | Blue | 350 |
| Example 3 | H3 | 4.6 | Blue | 330 |
| Example 4 | H4 | 5.5 | Blue | 430 |
| Example 5 | H5 | 5.6 | Blue | 440 |
| Example 6 | H6 | 5.6 | Blue | 440 |
| Example 7 | H7 | 5.2 | Blue | 400 |
| Example 8 | H8 | 4.9 | Blue | 420 |
| Example 9 | H9 | 4.7 | Blue | 410 |
| Example 10 | H10 | 4.8 | Blue | 420 |
| Example 11 | H11 | 5.0 | Blue | 390 |
| Example 12 | H12 | 5.4 | Blue | 410 |
| Example 13 | H1 | 4.8 | Blue | 360 |
| Example 14 | H4 | 5.4 | Blue | 440 |
| Comparative Example 1 | Comparative Compound 1 | 4.6 | Blue | 140 |
| Comparative Example 2 | Comparative Compound 2 | 3.9 | Blue | 160 |
| Comparative Example 3 | Comparative Compound 1 | 4.6 | Blue | 150 |

### Example 15 (Production of Organic EL Device)

A glass substrate with an ITO transparent electrode measuring mm wide by 75 mm long by 1.1 mm thick (manufactured by GEOMATEC Co., Ltd.) was subjected to ultrasonic cleaning in isopropyl alcohol for 5 minutes. After that, the substrate was subjected to UV ozone cleaning for 30 minutes.

The glass substrate with the transparent electrode line after the washing was mounted on a substrate holder of a vacuum deposition device. First, Compound H232 to be described below was formed into a film having a thickness of 60 nm on the surface on the side where the transparent electrode line was formed to cover the transparent electrode. The H232 film functions as a hole injecting layer. Compound H1 described above was formed into a film having a thickness of 20 nm on the H232 film. The film functions as a hole transporting layer. Further, Compound EM1 to be described below was deposited from the vapor and formed into a film having a thickness of 40 nm. Simultaneously with this formation, Amine Compound D1 having a styryl group to be described below, as a light emitting molecule, was deposited from the vapor in such a manner that a weight ratio between Compound EM1 and Amine Compound D1 would be 40:2. The film functions as a light emitting layer.

Alq to be described below was formed into a film having a thickness of 10 nm on the resultant film. The film functions as an electron injecting layer. After that, Li serving as a reducing dopant (Li source: manufactured by SAES Getters) and Alq were subjected to co-deposition. Thus, an Alq:Li film (having a thickness of 10 nm) was formed as an electron injecting layer (cathode). Metal Al was deposited from the vapor onto the Alq:Li film to form a metal cathode. Thus, an organic EL device was formed.

In addition, the current efficiency of the resultant organic EL device was measured, and the luminescent color of the device was observed. A current efficiency of 10 mA/cm² was calculated by measuring a luminance by using a CS1000 manufactured by Minolta. Further, the half lifetime of light emission in DC constant current driving at an initial luminance of 5,000 cd/m² and room temperature was measured. Table 2 shows the results.

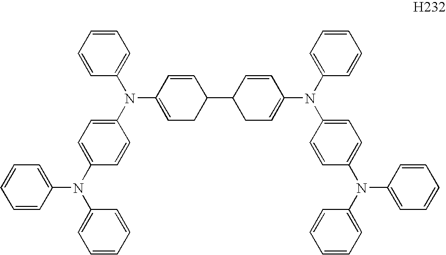

### Examples 16 and 17 (Production of Organic EL Devices)

An experiment and measurement were each performed in the same manner as in Example 15 except that a compound described in Table 2 was used instead of Compound H1 as a hole transporting material. Table 2 shows the results.

### Comparative Examples 4 and 5

An experiment and measurement were each performed in the same manner as in Example 15 except that Comparative Compound 1 or Comparative Compound 2 was used instead of Compound H1 as a hole transporting material. Table 2 shows the results.

### Example 18 (Production of Organic EL Device)

An experiment and measurement were each performed in the same manner as in Example 15 except that Arylamine Compound D2 shown above was used instead of Amine Compound D1 having a styryl group. Table 2 shows the results.

### Comparative Examples 6

An experiment and measurement were each performed in the same manner as in Example 18 except that Comparative Compound 1 shown above was used instead of Compound H1 as a hole transporting material. Table 2 shows the results.

### Examples 19 to 27 (Production of Organic EL Device)

An experiment and measurement were each performed in the same manner as in Example 15 except that a compound described in Table 2 was used instead of Compound H1 as a hole transporting material. Table 2 shows the results.

### Example 28 (Production of Organic EL Device)

An experiment and measurement were each performed in the same manner as in Example 19 except that Arylamine Compound D2 shown above was used instead of Amine Compound D1 having a styryl group. Table 2 shows the results.

TABLE 2

| | | Current | | |
|---|---|---|---|---|
| | Hole transporting | efficiency | Luminescent | Half lifetime |
| | material | (cd/A) | color | (h) |
| Example 15 | H1 | 5.1 | Blue | 370 |
| Example 16 | H2 | 5.0 | Blue | 360 |
| Example 17 | H3 | 4.8 | Blue | 330 |
| Example 18 | H1 | 5.1 | Blue | 380 |
| Example 19 | H4 | 5.9 | Blue | 310 |
| Example 20 | H5 | 6.1 | Blue | 320 |
| Example 21 | H6 | 6.0 | Blue | 330 |
| Example 22 | H7 | 5.6 | Blue | 310 |
| Example 23 | H8 | 4.9 | Blue | 430 |
| Example 24 | H9 | 4.8 | Blue | 420 |
| Example 25 | H10 | 4.9 | Blue | 430 |
| Example 26 | H11 | 5.0 | Blue | 400 |
| Example 27 | H12 | 5.5 | Blue | 310 |
| Example 28 | H4 | 5.9 | Blue | 320 |
| Comparative Example 4 | Comparative Compound 1 | 4.8 | Blue | 260 |
| Comparative Example 5 | Comparative Compound 2 | 4.3 | Blue | 210 |
| Comparative Example 6 | Comparative Compound 1 | 4.7 | Blue | 260 |

### Example 29 (Production of Organic EL Device)

An experiment and measurement were each performed in the same manner as in Example 1 except that: Acceptor Compound to be described below was formed into a film having a thickness of 10 nm between an anode and Compound H1 shown above; and the thickness of the film formed of Compound H1 shown above was changed to 50 nm.

As a result, the device showed a current efficiency of 4.3 cd/A, emitted blue light, and had a half lifetime of 310 hours.

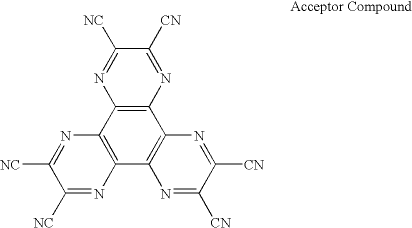

### Comparative Example 7

An experiment and measurement were each performed in the same manner as in Example 29 except that a Comparative compound 1 described above was used instead of Compound H1 as a hole transporting material. Table 2 shows the results.

As a result, the device showed a current efficiency of 4.1 cd/A, emitted blue light, and had a half lifetime of 90 hours.

### Example 30 (Production of Organic EL Device)

An experiment and measurement were each performed in the same manner as in Example 4 except that: Acceptor Compound used in Example 29 was formed into a film having a thickness of 10 nm between an anode and Compound H4 shown above; and the thickness of the film formed of Compound H4 shown above was changed to 50 nm.

As a result, the device showed a current efficiency of 4.9 cd/A, emitted blue light, and had a half lifetime of 380 hours.

### INDUSTRIAL APPLICABILITY

As described above in detail, the aromatic amine derivative of the present invention improves the efficiency of an organic EL device using the derivative, and its molecules hardly crystallize; furthermore, an organic EL device having a long lifetime can be produced in improved yield by incorporating the derivative into the organic thin film layer of the device.

1. An aromatic amine derivative represented by the following general formula (1): 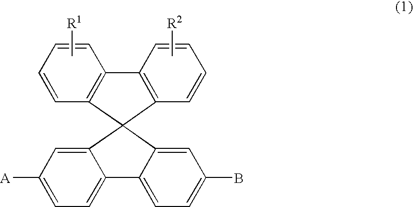
where R¹ and R² each independently represent a hydrogen atom, a substituted or unsubstituted aryl group having 6 to 50 ring atoms, or a substituted or unsubstituted alkyl group having 1 to 50 carbon atoms, and A and B are each independently represented by any one of the following general formulae (2) to (5) provided that A and B are different from each other: 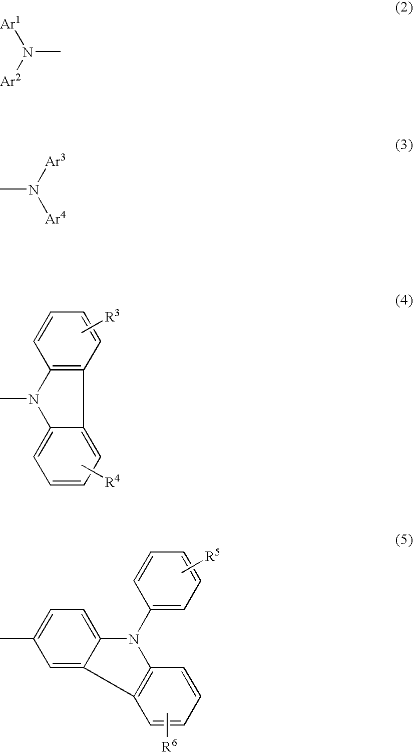
where Ar¹ to Ar⁴ each independently represent a substituted or unsubstituted aryl group having 6 to 50 ring atoms, and R³ to R⁶ each independently represent a hydrogen atom, a substituted or unsubstituted aryl group having 6 to 50 ring atoms, or a substituted or unsubstituted alkyl group having 1 to 50 carbon atoms.

2. The aromatic amine derivative according to claim 1, wherein A in the general formula (1) represents a substituent represented by the general formula (2), B in the general formula (1) represents a substituent represented by the general formula (3), and at least three of substituents represented by Ar¹ to Ar⁴ are different from one another.

3. The aromatic amine derivative according to claim 1, wherein A in the general formula (1) represents a substituent represented by the general formula (2), B in the general formula (1) represents a substituent represented by the general formula (3), and three of substituents represented by Ar¹ to Ar⁴ are identical to one another.

4. The aromatic amine derivative according to claim 1, wherein A in the general formula (1) represents a substituent represented by the general formula (2), B in the general formula (1) represents a substituent represented by the general formula (3), Ar¹ and Ar² represent the same substituent and Ar³ and Ar⁴ represent the same substituent.

5. The aromatic amine derivative according to claim 1, wherein A in the general formula (1) represents a substituent represented by the general formula (2), B in the general formula (1) represents a substituent represented by the general formula (3), Ar¹ and Ar² each represent a biphenyl group, and Ar³ and Ar⁴ each independently represent a substituent selected from a phenyl group, a biphenyl group, a naphthyl group, a terphenyl group, and a fluorenyl group.

6. The aromatic amine derivative according to claim 1, wherein A in the general formula (1) represents a substituent represented by the general formula (2) and B in the general formula (1) represents a substituent represented by the general formula (4).

7. The aromatic amine derivative according to claim 6, wherein Ar¹ and Ar2 in the general formula (2) each independently represent a substituent selected from a phenyl group, a biphenyl group, a naphthyl group, a terphenyl group, and a fluorenyl group.

8. The aromatic amine derivative according to claim 1, wherein A in the general formula (1) represents a substituent represented by the general formula (2) and B in the general formula (1) represents a substituent represented by the general formula (5).

9. The aromatic amine derivative according to claim 8, wherein Ar¹ and Ar² in the general formula (2) each independently represent a substituent selected from a phenyl group, a biphenyl group, a naphthyl group, a terphenyl group, and a fluorenyl group.

10. The aromatic amine derivative according to claim 1, wherein A in the general formula (1) represents a substituent represented by the general formula (4) and B in the general formula (1) represents a substituent represented by the general formula (5).

11. The aromatic amine compound according to any one of claims 1 to 10, comprising a material for an organic electroluminescent device.

12. The aromatic amine compound according to any one of claims 1 to 10, comprising a hole transporting material for an organic electroluminescent device.

13. An organic electroluminescent device, comprising an organic thin film layer formed of one or a plurality of layers including at least a light emitting layer and interposed between a cathode and an anode, wherein at least one layer of the organic thin film layer contains the aromatic amine derivative according to any one of claims 1 to 10 alone or as a component of a mixture.

14. The organic electroluminescent device according to claim 13, wherein the organic thin film layer has a hole transporting layer and the aromatic amine derivative according to any one of claims 1 to 10 is contained in the hole transporting layer.

15. The organic electroluminescence device according to claim 13, wherein the organic thin film layer has a plurality of hole transporting layers, and a layer in direct contact with the light emitting layer contains the aromatic amine derivative according to any one of claims 1 to 10.

16. The organic electroluminescence device according to claim 13, wherein the organic thin film layer has a hole injecting layer, and the hole injecting layer contains the aromatic amine derivative according to any one of claims 1 to 10.

17. The organic electroluminescent device according to any one of claims 13 to 16, further comprising a styrylamine compound and/or an arylamine compound in a light emitting layer.

18. The organic electroluminescence device according to any one of claims 13 to 17, wherein the organic thin film layer has a plurality of hole injecting and transporting layers, and at least one of the layers comprises a layer containing an acceptor material.

19. The organic electroluminescent device according to any one of claims 13 to 18, which emits blue-based light.
